(19) United States
(12) Patent Application Publication
(10) Pub. No.: US 2025/0136583 A1
(43) Pub. Date: May 1, 2025

(54) NOVEL SALT FORMS OF ISOCHROMAN-IMIDAZOLE STRUCTURED ALPHA-2A ADRENOCEPTOR AGONIST

(71) Applicant: ORION CORPORATION, Espoo (FI)

(72) Inventors: Anna HAAPALINNA, Espoo (FI); Kristo HAKALA, Espoo (FI); Tero LINNANEN, Naantali (FI); Antti POHJAKALLIO, Espoo (FI); Mihaela POP, Amsterdam (NL)

(21) Appl. No.: 18/835,483

(22) PCT Filed: Feb. 3, 2023
(86) PCT No.: PCT/FI2023/050070

§ 371 Date: Aug. 2, 2024

(30) Foreign Application Priority Data
Feb. 4, 2022 (FI) 20225095

(51) Int. Cl. C07D 405/04; A61K 31/4178

(52) U.S. Cl. CPC C07D 405/04; A61K 31/4178

(57) ABSTRACT

The present disclosure relates to novel salts, particularly crystalline salts of 2-(5-methoxyisochroman-1-yl)-4,5-dihydro-1H-imidazole (I) which are suitable for use in the manufacture of pharmaceutical compositions. Compound (I) is selective alpha2A adrenoceptor agonist and is useful in the treatment of anxiety, and for use as a sedative or analgesic agent, and other diseases were alpha2A agonism is desired.

### TECHNICAL FIELD

The present disclosure relates to a novel salts of 2-(5-methoxyisochroman-1-yl)-4,5-dihydro-1H-imidazole (I) and to preparation thereof. Furthermore, the disclosure relates to pharmaceutical composition comprising such novel salts.

### BACKGROUND OF THE INVENTION

Tasipimidine is the International Nonproprietary Name (INN) of the compound 2-(5-methoxyisochroman-1-yl)-4,5-dihydro-1H-imidazole represented by the following structural formula (I):

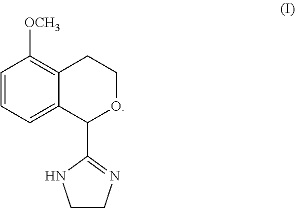 [SMILES: COc1cccc2c1CCOC2C1=NCCN1]

Tasipimidine and derivatives thereof have been disclosed in WO 2013/150173. While WO 2013/150173 generally mentions that the compound of formula (I), among others, can form a pharmaceutically acceptable salt with both organic and inorganic bases and acids, only the hydrochloride salt, sulfate salt and hemifumarate salts have been prepared, but no specific crystalline forms of the salts are mentioned. Tasipimidine exhibits agonistic activities on adrenergic alpha2 receptors, especially on alpha2A receptor, and can thus be used in the treatment of a disorder, condition or disease where an alpha2A agonist is indicated to be useful, for example, for use as a sedative or analgesic agent, and for use in the treatment of anxiety. Tasipimidine and sulfate salt thereof may be prepared using the method described, for example, in WO 2019/106238.

Typically, to enable the efficient development of solid dosage forms, a form of the active ingredient is sought that has a balance of desired properties, such as crystallinity, lack of polymorphism, high melting point, solid-state stability, compressibility and lack of hygroscopicity together with satisfactory solubility. For example, it is desired that a form of the active ingredient, which has the requisite bioavailability, also has sufficient stability such that it does not degrade or convert during manufacture or storage of the pharmaceutical composition to a different form, which has different properties.

Thus, one or more forms of compound (I) are desired having properties and stability that allow a large scale manufacture of marketable pharmaceutical product.

The existence and properties of individual salts are inherently unpredictable. Hence although numerous acids are available to try as alternatives, the skilled person cannot predict which, if any, is likely to provide a salt having physical and/or chemical properties to make it suitable to be included into a pharmaceutical composition. Salt preparation with various acids, such as naphthalene-1,5-disulfonic, cyclamic, ethanesulfonic, ethane-1,2-disulfonic, p-toluenesulfonic, methanesulfonic, naphthalene-2-sulfonic, benzenesulfonic, oxalic, saccharin, L-glutamic, maleic, phosphoric, malonic, gentisic, L-tartaric, fumaric, citric, D-glucoronic, glycolic, L-malic, hippuric, D,L-lactic, L-ascorbic, benzoic, succinic, glutaric, adipic, acetic, hydrobromic, 2-ketoglutaric, L-aspartic, mucic, 1-hydroxy-2-napthoic, pamoic, (+)-camphoric, and hydrochloric acid was attempted. Compound (I) showed its ability to crystallize in several salt forms with different acids, but in some cases the formed salt was chemically or physically unstable, and in some cases, no salt was formed at all.

### SUMMARY OF THE INVENTION

It has now been found that 2-(5-methoxyisochroman-1-yl)-4,5-dihydro-1H-imidazole (I), may exist in one or more crystalline salt forms having the necessary properties, including stability and processability, to enable their use in large scale manufacture of pharmaceutical products.

Accordingly, the present disclosure relates to a salt of compound (I) with naphthalene-1,5-disulfonic acid, ethane-1,2-disulfonic acid, oxalic acid, saccharin, maleic acid, malonic acid, L-tartaric acid, fumaric acid, glycolic acid, glutaric acid, adipic acid, hydrobromic acid, mucic acid, 1-hydroxy-2-napthoic acid, and hydrochloric acid.

Especially, the present disclosure relates to naphthalene-1,5-disulfonic acid, ethane-1,2-disulfonic acid, oxalic acid, saccharin, maleic acid, malonic acid, L-tartaric acid, glycolic acid, glutaric acid, adipic acid, hydrobromic acid, mucic acid, and 1-hydroxy-2-napthoic acid salt of compound (I).

In one aspect, the present disclosure relates to the above salts in crystalline form.

In particular, the present disclosure relates to napthalene-1,5-disulfonic acid, ethane-1,2-disulfonic acid, oxalic acid, and hydrobromic acid salt of compound (I). These salts are crystalline and exhibit good chemical stability. In addition, such salts are easy to handle and process, and therefore suitable for the manufacture of various pharmaceutical dosage forms.

The disclosure also relates to pharmaceutical composition comprising any of the above salts of 2-(5-methoxyisochroman-1-yl)-4,5-dihydro-1H-imidazole (I), and to use of the novel salts in the treatment of anxiety, and for use as a sedative or analgesic agent, and other diseases were alpha2A agonism is desired.

The foregoing as well as other feature and advantages of the present teachings will be more fully understood from the following description and claims.

### BRIEF DESCRIPTION OF THE DRAWINGS

FIG. 1 shows the X-ray powder diffraction (XRPD) pattern of the crystalline form 1 of naphtalene-1,5-disulfonic acid salt of compound (I).

FIG. 2 shows the differential scanning calorimetry (DSC) thermogram of the crystalline form 1 of naphtalene-1,5-disulfonic acid salt of compound (I).

FIG. 3 shows the X-ray powder diffraction (XRPD) pattern of the crystalline form 1 of ethane-1,2-disulfonic acid salt of compound (I).

FIG. 4 shows the differential scanning calorimetry (DSC) thermogram of the crystalline form 1 of ethane-1,2-disulfonic acid salt of compound (I).

FIG. 5 shows the X-ray powder diffraction (XRPD) pattern of the crystalline form 1 of oxalic acid salt of compound (I).

FIG. 6 shows the differential scanning calorimetry (DSC) thermogram of the crystalline form 1 of oxalic acid salt of compound (I).

FIG. 7 shows the X-ray powder diffraction (XRPD) pattern of the crystalline form 1 of saccharin salt of compound (I).

FIG. 8 shows the differential scanning calorimetry (DSC) thermogram of the crystalline form 1 of saccharin salt of compound (I).

FIG. 9 shows the X-ray powder diffraction (XRPD) pattern of the crystalline form 2 of saccharin salt of compound (I).

FIG. 10 shows the differential scanning calorimetry (DSC) thermogram of the crystalline form 2 of saccharin salt of compound (I).

FIG. 11 shows the X-ray powder diffraction (XRPD) pattern of the crystalline form 1 of maleic acid salt of compound (I).

FIG. 12 shows the differential scanning calorimetry (DSC) thermogram of the crystalline form 1 of maleic acid salt of compound (I).

FIG. 13 shows the X-ray powder diffraction (XRPD) pattern of the crystalline form 1 of malonic acid salt of compound (I).

FIG. 14 shows the differential scanning calorimetry (DSC) thermogram of the crystalline form 1 of malonic acid salt of compound (I).

FIG. 15 shows the X-ray powder diffraction (XRPD) pattern of the crystalline form 1 of L-tartaric acid salt of compound (I).

FIG. 16 shows the differential scanning calorimetry (DSC) thermogram of the crystalline form 1 of L-tartaric acid salt of compound (I).

FIG. 17 shows the X-ray powder diffraction (XRPD) pattern of the crystalline form 2 of L-tartaric acid salt of compound (I).

FIG. 18 shows the differential scanning calorimetry (DSC) thermogram of the crystalline form 2 of L-tartaric acid salt of compound (I).

FIG. 19 shows the X-ray powder diffraction (XRPD) pattern of the crystalline form 1 of fumaric acid salt of compound (I).

FIG. 20 shows the differential scanning calorimetry (DSC) thermogram of the crystalline form 1 of fumaric acid salt of compound (I).

FIG. 21 shows the X-ray powder diffraction (XRPD) pattern of the crystalline form 2 of fumaric acid salt of compound (I).

FIG. 22 shows the differential scanning calorimetry (DSC) thermogram of the crystalline form 2 of fumaric acid salt of compound (I).

FIG. 23 shows the X-ray powder diffraction (XRPD) pattern of the crystalline form 1 of glycolic acid salt of compound (I).

FIG. 24 shows the differential scanning calorimetry (DSC) thermogram of the crystalline form 1 of glycolic acid salt of compound (I).

FIG. 25 shows the X-ray powder diffraction (XRPD) pattern of the crystalline form 1 of glutaric acid salt of compound (I).

FIG. 26 shows the differential scanning calorimetry (DSC) thermogram of the crystalline form 1 of glutaric acid salt of compound (I).

FIG. 27 shows the X-ray powder diffraction (XRPD) pattern of the crystalline form 1 of adipic acid salt of compound (I).

FIG. 28 shows the differential scanning calorimetry (DSC) thermogram of the crystalline form 1 of adipic acid salt of compound (I).

FIG. 29 shows the X-ray powder diffraction (XRPD) pattern of the crystalline form 1 of hydrobromic acid salt of compound (I).

FIG. 30 shows the differential scanning calorimetry (DSC) thermogram of the crystalline form 1 of hydrobromic acid salt of compound (I).

FIG. 31 shows the X-ray powder diffraction (XRPD) pattern of the crystalline form 1 of mucic acid salt of compound (I).

FIG. 32 shows the differential scanning calorimetry (DSC) thermogram of the crystalline form 1 of mucic acid salt of compound (I).

FIG. 33 shows the X-ray powder diffraction (XRPD) pattern of the crystalline form 1 of 1-hydroxy-2-naphthoic acid salt of compound (I).

FIG. 34 shows the differential scanning calorimetry (DSC) thermogram of the crystalline form 1 of 1-hydroxy-2-naphthoic acid salt of compound (I).

FIG. 35 shows the X-ray powder diffraction (XRPD) pattern of the crystalline form 1 of hydrochloric acid salt of compound (I).

FIG. 36 shows the differential scanning calorimetry (DSC) thermogram of the crystalline form 1 of hydrochloric acid salt of compound (I).

### DETAILED DESCRIPTION OF THE INVENTION

The present disclosure relates to novel salts of 2-(5-methoxyisochroman-1-yl)-4,5-dihydro-1H-imidazole (I). In particular, the present disclosure relates to salts of compound (I) in crystalline form.

Novel salts of compound (I) have been characterized by an X-ray powder diffraction (XRPD) studies.

Salt with Naphtalene-1,5-Disulfonic Acid

Accordingly, in one embodiment, the present disclosure relates to a salt of 2-(5-methoxyisochroman-1-yl)-4,5-dihydro-1H-imidazole (I) with naphtalene-1,5-disulfonic acid, particularly in crystalline form.

The salt with naphtalene-1,5-disulfonic acid can be prepared, for example, by dissolving compound (I) and naphtalene-1,5-disulfonic acid, for example, in equivalent molar amounts, in a suitable solvent followed by anti-solvent addition. Suitable solvents include, for example, acetone, ethanol and 2-propanol; such as acetone. Naphtalene-1,5-disulfonic acid may be dissolved in water before being added to the mixture. Anti-solvent can be added to the solution, for example in 4 steps, at about room temperature under constant stirring. Suitable anti-solvents include, for example, water, tert-butyl methyl ether (TBME) and heptane; such as water. After the anti-solvent addition, the mixture is aging at about 5° C. for several hours, for example, about 24-72 hours. The precipitated salt can then be isolated, for example, by filtration or decantation, followed by solvent evaporation at reduced pressure, for example under vacuum at about 25-45° C. The product is then recrystallized by dissolving it in a suitable solvent, for example, ethyl acetate or TBME at elevated temperature, for example, at about 50° C. The obtained solution is allowed to cool, for example, to about room temperature followed by slow solvent evaporation in open vials. The precipitated crystalline salt can then be isolated, for example, by decantation.

The salt of compound (I) with naphtalene-1,5-disulfonic acid appears to precipitate in a single crystalline form, named herein as crystalline form 1. No other crystalline forms have been found for naphtalene-1,5-disulfonic acid salt.

In one embodiment, the present disclosure relates to crystalline form 1 of the salt of compound (I) with naphtalene-1,5-disulfonic acid having an X-ray powder diffraction pattern comprising characteristic peaks at about 18.6, 19.1, 21.5, 22.4 and 23.9 degrees 2-theta.

In one embodiment, the present disclosure relates to crystalline form 1 of the salt of compound (I) with naphtalene-1,5-disulfonic acid having an X-ray powder diffraction pattern comprising characteristic peaks at about 8.2, 8.5, 9.6, 10.2, 14.2, 15.7, 16.2, 17.6, 18.2, 18.6, 19.1, 21.5, 22.4 and 23.9 degrees 2-theta.

In one embodiment, the crystalline form 1 of the salt of compound (I) with naphtalene-1,5-disulfonic acid is further characterized by an X-ray powder diffraction pattern as depicted in FIG. 1.

Salt with Ethane-1,2-Disulfonic Acid

In one embodiment, the present disclosure relates to a salt of 2-(5-methoxyisochroman-1-yl)-4,5-dihydro-1H-imidazole (I) with ethane-1,2-disulfonic acid, particularly in crystalline form.

The salt with ethane-1,2-disulfonic acid can be prepared, for example, by dissolving compound (I) and ethane-1,2-disulfonic acid, for example, in equivalent molar amounts, in a suitable solvent followed by anti-solvent addition. Suitable solvents include, for example, acetone, ethanol and 2-propanol; such as 2-propanol. Ethane-1,2-disulfonic acid may be dissolved in water before being added to the mixture. Anti-solvent can be added to the solution, for example in 2-4 steps, at about room temperature under constant stirring.

Suitable anti-solvents include, for example, water, TBME and heptane; such as heptane. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with ethane-1,2-disulfonic acid appears to precipitate in a single crystalline form, named herein as crystalline form 1. No other crystalline forms have been found for ethane-1,2-disulfonic acid salt.

In one embodiment, the present disclosure relates to crystalline form 1 of the salt of compound (I) with ethane-1,2-disulfonic acid having an X-ray powder diffraction pattern comprising characteristic peaks at about 13.5, 14.5, 16.0, 20.3 and 22.8 degrees 2-theta.

In one embodiment, the present disclosure relates to crystalline form 1 of the salt of compound (I) with ethane-1,2-disulfonic acid having an X-ray powder diffraction pattern comprising characteristic peaks at about 9.9, 12.1, 13.5, 14.5, 16.0, 19.9, 20.1, 20.3, 21.7 and 22.8 degrees 2-theta.

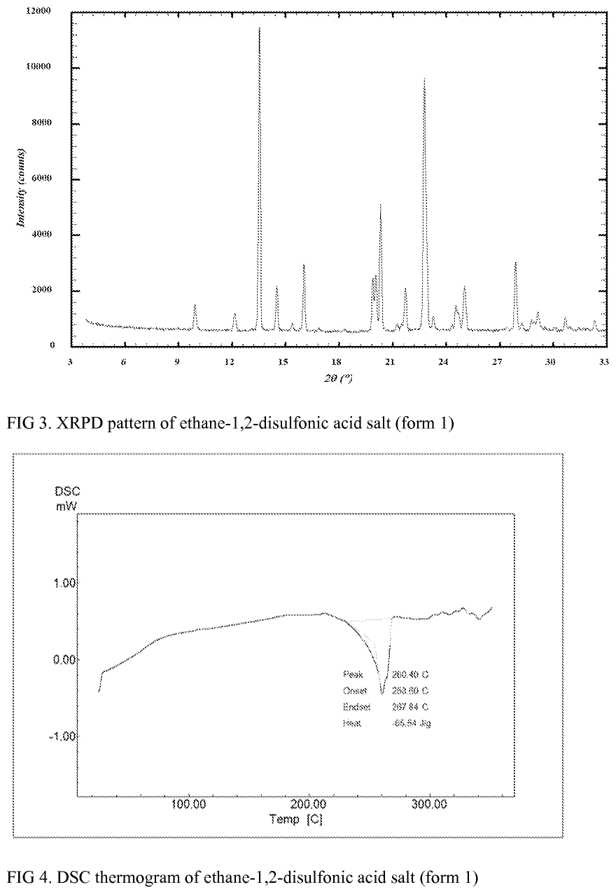

In one embodiment, the crystalline form 1 of the salt of compound (I) with ethane-1,2-disulfonic acid is further characterized by an X-ray powder diffraction pattern as depicted in FIG. 3.

Salt with Oxalic Acid

In one embodiment, the present disclosure relates to a salt of 2-(5-methoxyisochroman-1-yl)-4,5-dihydro-1H-imidazole (I) with oxalic acid, particularly in crystalline form.

The salt with oxalic acid can be prepared, for example, by dissolving compound (I) and oxalic acid, for example, in equivalent molar amounts, in a suitable solvent at room or elevated temperature, cooling the mixture, and isolating the crystalline product. Suitable solvents include, for example, ethanol and acetone; such as acetone. Oxalic acid may optionally be dissolved in acetone before being added to the mixture. The mixture can be heated, for example, to the temperature about 50-70° C., suitably to about 60° C. The obtained solution is stirred and allowed to cool, for example to about room temperature over several hours, for example, about 2 hours. The mixture can be further cooled to about 5° C. followed by aging at about 5° C. for several hours, for example, about 24-72 hours. The precipitated crystalline salt can then be isolated, for example, by centrifugation and decantation, followed by slow solvent evaporation at about room temperature in open vials.

The salt of compound (I) with oxalic acid appears to precipitate in a single crystalline form, named herein as crystalline form 1. No other crystalline forms have been found for oxalic acid salt.

In one embodiment, the present disclosure relates to crystalline form 1 of the salt of compound (I) with oxalic acid having an X-ray powder diffraction pattern comprising characteristic peaks at about 14.3, 17.0, 17.6, 19.8, 22.3 and 23.5 degrees 2-theta.

In one embodiment, the present disclosure relates to crystalline form 1 of the salt of compound (I) with oxalic acid having an X-ray powder diffraction pattern comprising characteristic peaks at about 5.9, 10.8, 11.7, 12.1, 14.3, 17.0, 17.6, 19.8, 22.3 and 23.5 degrees 2-theta.

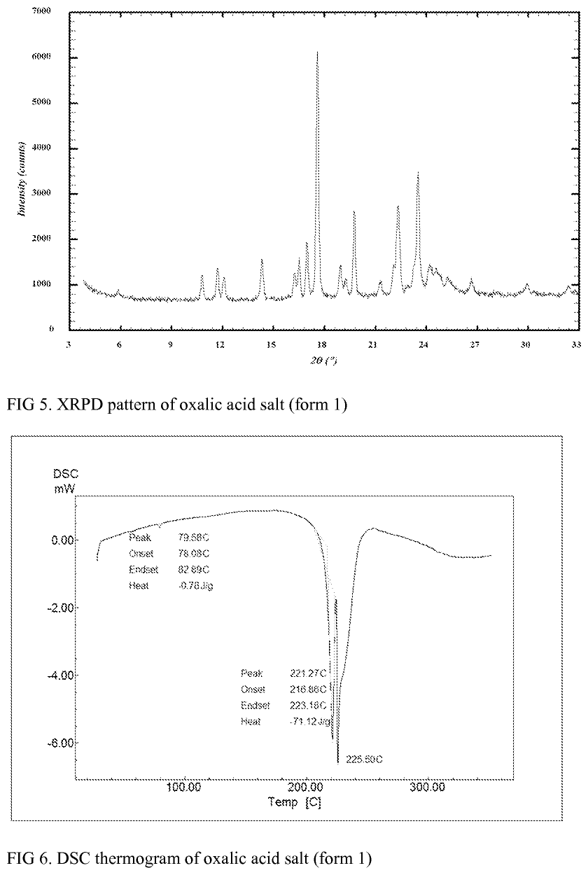

In one embodiment, the crystalline form 1 of the salt of compound (I) with oxalic acid is further characterized by an X-ray powder diffraction pattern as depicted in FIG. 5.

Salt with Saccharin

In one embodiment, the present disclosure relates to a salt of 2-(5-methoxyisochroman-1-yl)-4,5-dihydro-1H-imidazole (I) with saccharin, particularly in crystalline form.

The salt with saccharin has been found to exist in two crystalline forms, named herein as crystalline forms 1 and 2. However, on storage at elevated temperature and relative humidity (40° C., 75% RH for 3 weeks) crystalline form 1 exhibited partially conversion into form 2.

The saccharin salt crystalline form 1 can be prepared, for example, by dissolving compound (I) and saccharin, for example, in equivalent molar amounts, in a suitable solvent at room or elevated temperature, cooling the mixture, and isolating the crystalline product. Suitable solvents include, for example, ethanol and acetone; such as ethanol. Saccharin may optionally be dissolved in ethanol before being added to the mixture. The mixture can be heated, for example, to the temperature about 50-70° C., suitably to about 60° C. The obtained solution is stirred and allowed to cool, for example to about room temperature over several hours, for example, about 2 hours. The mixture can be further cooled to about 5° C. followed by aging at about 5° C. for several hours, for example, about 24-72 hours. The precipitated salt can then be isolated, for example, by slow solvent evaporation at about room temperature in open vials. The product may then be recrystallized by dissolving it in a suitable solvent, for example, ethylacetate at slightly elevated temperature, for example, at about 45-55° C. The recrystallization may be repeated with TBME as a solvent. The obtained solution is allowed to cool, for example, to about room temperature followed by slow solvent evaporation in open vials to yield saccharin salt in crystalline form 1.

In one embodiment, the present disclosure relates to crystalline form 1 of the salt of compound (I) with saccharin having an X-ray powder diffraction pattern comprising characteristic peaks at about 9.4, 13.5, 17.1, 18.8 and 21.5 degrees 2-theta

In one embodiment, the present disclosure relates to crystalline form 1 of the salt of compound (I) with saccharin having an X-ray powder diffraction pattern comprising characteristic peaks at about 9.4, 13.5, 15.6, 16.0, 17.1, 18.8, 19.5, 20.5, 21.5 and 23.0 degrees 2-theta.

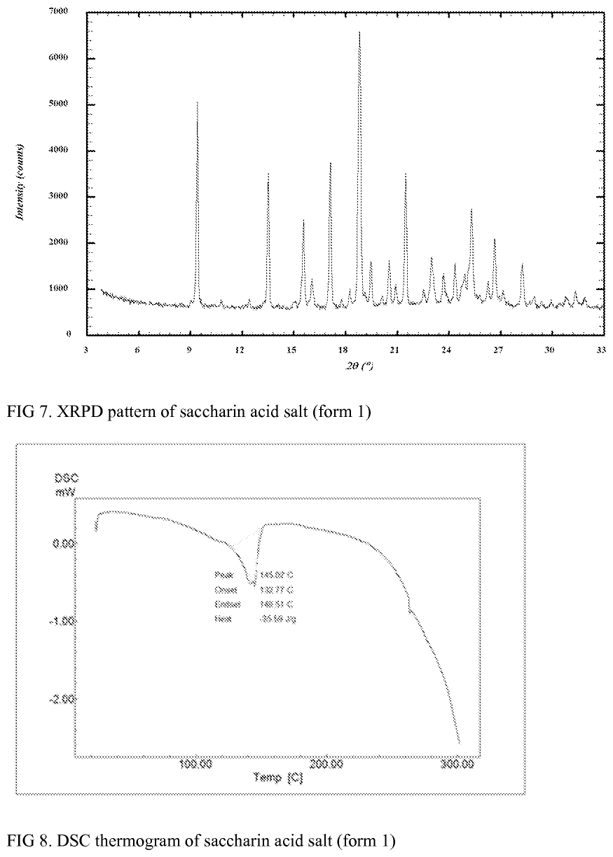

In one embodiment, the crystalline form 1 of the salt of compound (I) with saccharin is further characterized by an X-ray powder diffraction pattern as depicted in FIG. 7

The saccharin salt crystalline form 2 can be prepared, for example, by dissolving compound (I) and saccharin, for example, in equivalent molar amounts, in a suitable solvent followed by anti-solvent addition. Suitable solvents include, for example, acetone, ethanol and 2-propanol; such as ethanol. Saccharin may be dissolved in ethanol before being added to the mixture. Anti-solvent can be added to the solution, for example in 4 steps, at about room temperature under constant stirring. Suitable anti-solvents include, for example, water, TBME and heptane; such as TBME. After the anti-solvent addition, the mixture is aging at about 5° C. for several hours, for example, about 24-72 hours. The precipitated saccharin salt in crystalline form 2 can then be isolated, for example, by filtration or decantation, followed by slow solvent evaporation at about room temperature in open vials.

In one embodiment, the present disclosure relates to crystalline form 2 of the salt of compound (I) with saccharin having an X-ray powder diffraction pattern comprising characteristic peaks at about 14.1, 15.1, 15.8, 17.7 and 19.1 degrees 2-theta.

In one embodiment, the present disclosure relates to crystalline form 2 of the salt of compound (I) with saccharin having an X-ray powder diffraction pattern comprising characteristic peaks at about 7.5, 8.5, 9.2, 14.1, 15.1, 15.8, 16.2, 16.3, 17.7, 18.7 and 19.1, degrees 2-theta.

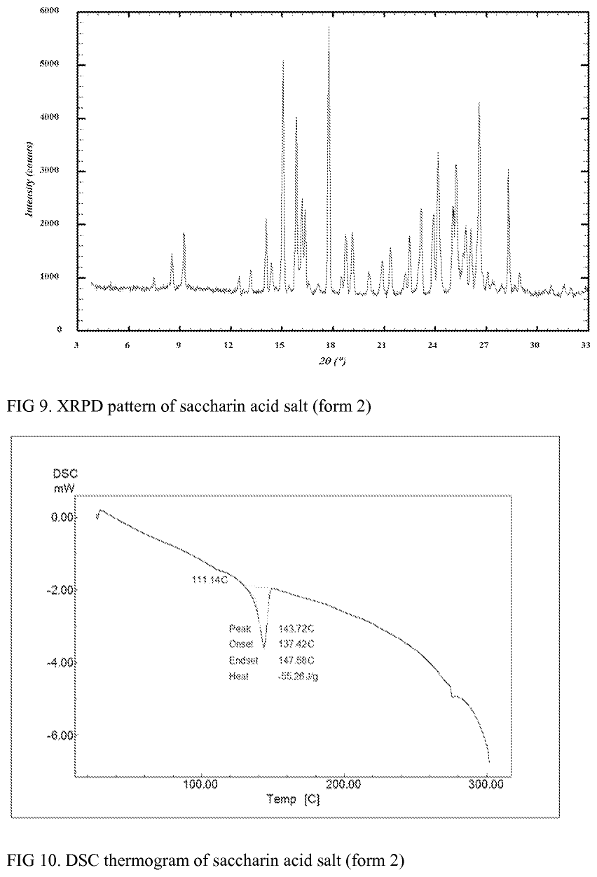

In one embodiment, the crystalline form 2 of the salt of compound (I) with saccharin is further characterized by an X-ray powder diffraction pattern as depicted in FIG. 9.

Salt with Maleic Acid

In one embodiment, the present disclosure relates to a salt of 2-(5-methoxyisochroman-1-yl)-4,5-dihydro-1H-imidazole (I) with maleic acid, particularly in crystalline form.

The salt with maleic acid can be prepared, for example, by dissolving compound (I) and maleic acid, for example, in equivalent molar amounts, in a suitable solvent followed by anti-solvent addition. Suitable solvents include, for example, acetone, ethanol and 2-propanol; such as 2-propanol. Maleic acid may be dissolved in 2-propanol before being added to the mixture. Anti-solvent can be added to the solution, for example in 4 steps, at about room temperature under constant stirring. Suitable anti-solvents include, for example, water, TBME and heptane; such as heptane. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with maleic acid appears to precipitate in a single crystalline form, named herein as crystalline form 1. No other crystalline forms have been found for maleic acid salt.

In one embodiment, the present disclosure relates to crystalline form 1 of the salt of compound (I) with maleic acid having an X-ray powder diffraction pattern comprising characteristic peaks at about 10.2, 11.6, 16.8, 17.4 and 19.3 degrees 2-theta.

In one embodiment, the present disclosure relates to crystalline form 1 of the salt of compound (I) with maleic acid having an X-ray powder diffraction pattern comprising characteristic peaks at about 10.2, 11.6, 14.3, 16.8, 17.1, 17.4, 19.3, 22.0, 22.3, 22.4, 23.3, 23.9 and 24.1 degrees 2-theta.

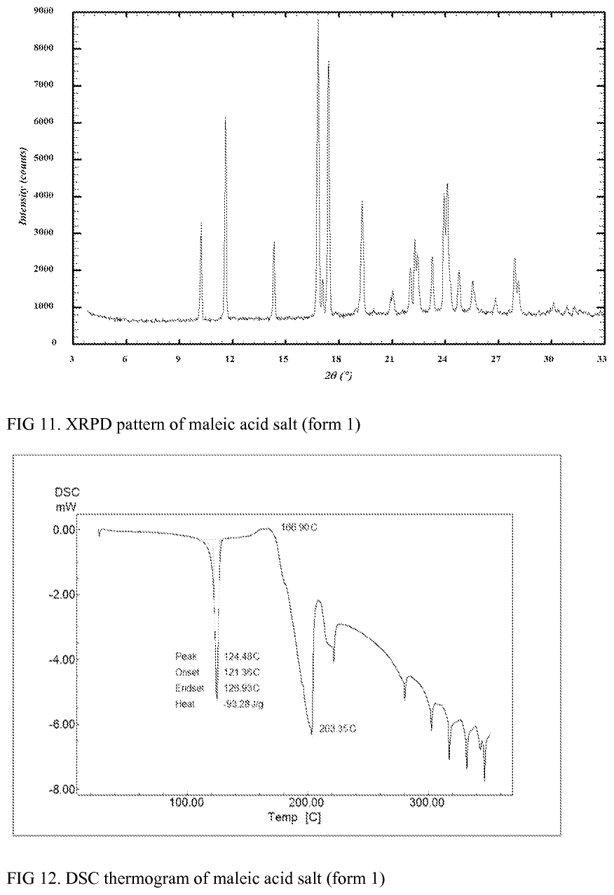

In one embodiment, the crystalline form 1 of the salt of compound (I) with maleic acid is further characterized by an X-ray powder diffraction pattern as depicted in FIG. 11.

Salt with Malonic Acid

In one embodiment, the present disclosure relates to a salt of 2-(5-methoxyisochroman-1-yl)-4,5-dihydro-1H-imidazole (I) with malonic acid, particularly in crystalline form.

The salt with malonic acid can be prepared, for example, by dissolving compound (I) and malonic acid, for example, in equivalent molar amounts, in a suitable solvent followed by anti-solvent addition. Suitable solvents include, for example, acetone, ethanol and 2-propanol; such as ethanol. Maleic acid may be dissolved in ethanol before being added to the mixture. Anti-solvent can be added to the solution, for example in 4 steps, at about room temperature under constant stirring. Suitable anti-solvents include, for example, water, TBME and heptane; such as TBME. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with malonic acid appears to precipitate in a single crystalline form, named herein as crystalline form 1. No other crystalline forms have been found for malonic acid salt.

In one embodiment, the present disclosure relates to crystalline form 1 of the salt of compound (I) with malonic acid having an X-ray powder diffraction pattern comprising characteristic peaks at about 10.1, 10.8, 14.1, 16.3 and 17.4 degrees 2-theta.

In one embodiment, the present disclosure relates to crystalline form 1 of the salt of compound (I) with malonic acid having an X-ray powder diffraction pattern comprising characteristic peaks at about 10.1, 10.4, 10.8, 13.7, 14.1, 16.3, 17.4, 21.8, 24.0, 24.2 and 24.7 degrees 2-theta.

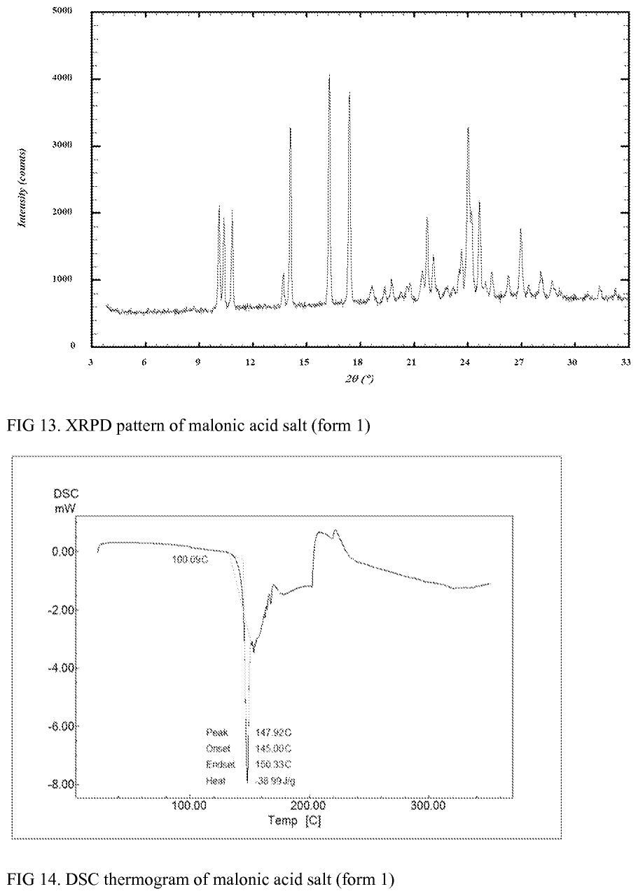

In one embodiment, the crystalline form 1 of the salt of compound (I) with malonic acid is further characterized by an X-ray powder diffraction pattern as depicted in FIG. 13.

Salt with L-Tartaric Acid

In one embodiment, the present disclosure relates to a salt of 2-(5-methoxyisochroman-1-yl)-4,5-dihydro-1H-imidazole (I) with L-tartaric acid, particularly in crystalline form.

The salt with L-tartaric acid has been found to exist in two crystalline forms, named herein as crystalline forms 1 and 2. However, on storage at elevated temperature and relative humidity (40° C., 75% RH for 3 weeks) crystalline form 2 turned into form 1.

L-tartaric acid salt crystalline form 1 can be prepared, for example, by dissolving compound (I) and L-tartaric acid, for example, in equivalent molar amounts, in a suitable solvent followed by anti-solvent addition. Suitable solvents include, for example, acetone, ethanol and 2-propanol; such as ethanol. L-tartaric acid may be dissolved in ethanol before being added to the mixture. Anti-solvent can be added to the solution, for example in 4 steps, at about room temperature under constant stirring. Suitable anti-solvents include, for example, water, TBME and heptane; such as TBME. After the anti-solvent addition, the mixture is aging at about 5° C. for several hours, for example, about 24-72 hours. The precipitated salt can then be isolated, for example, by filtration or decantation, followed by slow solvent evaporation at about room temperature. The product may then be recrystallized by dissolving it in a suitable solvent, for example, ethyl acetate or TBME at slightly elevated temperature, for example, at about 45-55° C. The obtained solution is allowed to cool, for example, to about room temperature followed by slow solvent evaporation in open vials and further drying under vacuum. The obtained white powder is exposed to about 40° C. and 75% relative humidity for about 3 weeks to yield L-tartaric acid salt in crystalline form 1.

In one embodiment, the present disclosure relates to crystalline form 1 of the salt of compound (I) with L-tartaric acid having an X-ray powder diffraction pattern comprising characteristic peaks at about 11.8, 12.5, 12.9, 13.4 and 14.0 degrees 2-theta.

In one embodiment, the present disclosure relates to crystalline form 1 of the salt of compound (I) with L-tartaric acid having an X-ray powder diffraction pattern comprising characteristic peaks at about 10.1, 11.8, 12.5, 12.9, 13.4, 14.0, 18.8, 19.3 and 20.8 degrees 2-theta.

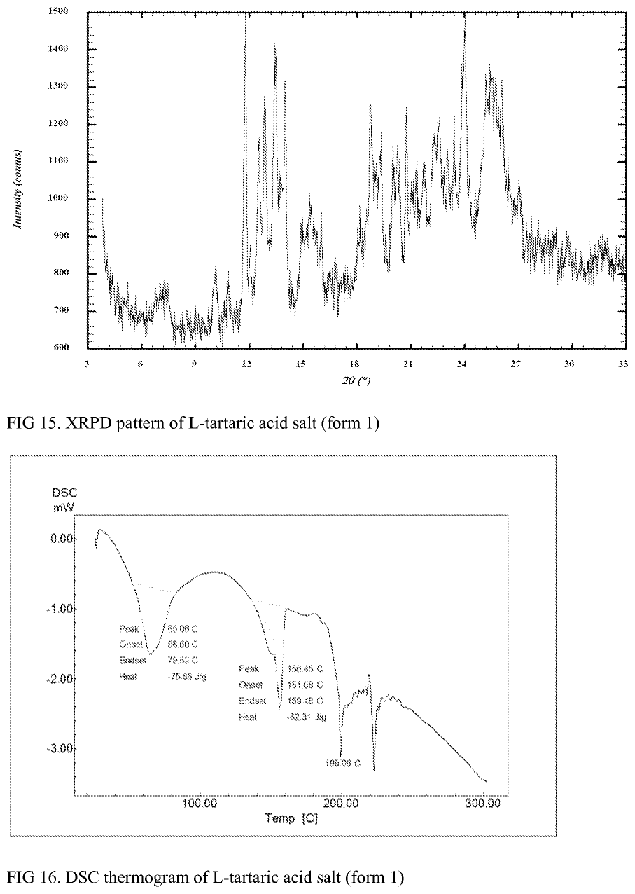

In one embodiment, the crystalline form 1 of the salt of compound (I) with L-tartaric acid is further characterized by an X-ray powder diffraction pattern as depicted in FIG. 15.

L-tartaric acid salt crystalline form 2 can be prepared, for example, by dissolving compound (I) and L-tartaric acid, for example, in equivalent molar amounts, in a suitable solvent followed by anti-solvent addition. Suitable solvents include, for example, acetone, ethanol and 2-propanol, such as ethanol. L-tartaric acid may be dissolved in ethanol before being added to the mixture. Anti-solvent can be added to the solution, for example in 4 steps, at about room temperature under constant stirring. Suitable anti-solvents include, for example, water, TBME and heptane, such as TBME. After the anti-solvent addition, the mixture is aging at about 5° C. for several hours, for example, about 24-72 hours. The precipitated salt can then be isolated, for example, by filtration or decantation, followed by slow solvent evaporation at about room temperature. The product is then recrystallized by dissolving it in a suitable solvent, for example, ethyl acetate or TBME at slightly elevated temperature, for example, at about 45-55° C. The obtained solution is allowed to cool, for example, to about room temperature followed by slow solvent evaporation in open vials to yield L-tartaric acid salt in crystalline form 2.

In one embodiment, the present disclosure relates to crystalline form 2 of the salt of compound (I) with L-tartaric acid having an X-ray powder diffraction pattern comprising characteristic peaks at about 7.2, 13.5, 21.1, 24.6 and 25.2 degrees 2-theta.

In one embodiment, the present disclosure relates to crystalline form 2 of the salt of compound (I) with L-tartaric acid having an X-ray powder diffraction pattern comprising characteristic peaks at about 7.2, 11.3, 12.0, 12.6, 13.5, 15.0, 15.2, 15.6, 15.8, 16.4, 17.1, 19.1, 21.1, 24.6 and 25.2 degrees 2-theta.

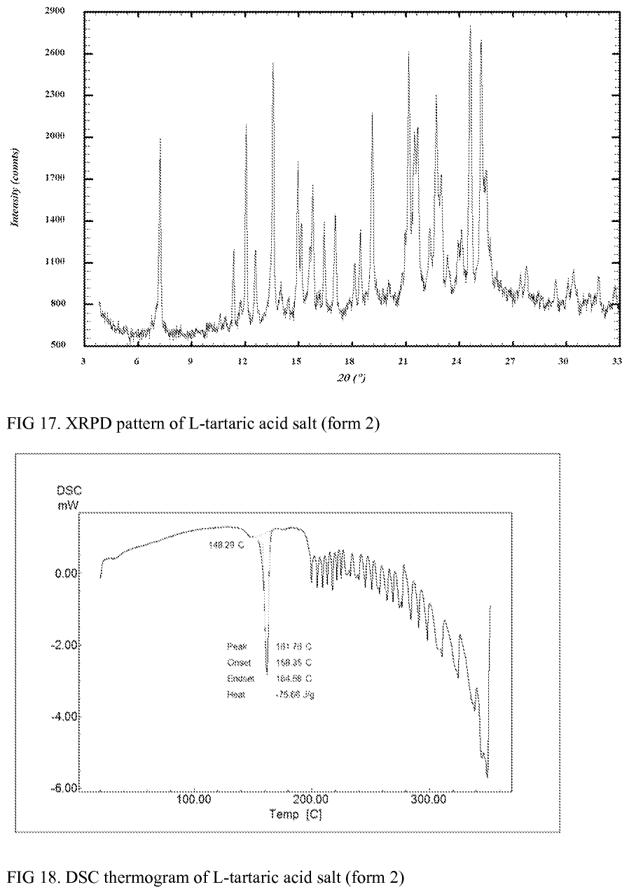

In one embodiment, the crystalline form 2 of the salt of compound (I) with L-tartaric acid is further characterized by an X-ray powder diffraction pattern as depicted in FIG. 17.

Salt with Fumaric Acid

In one embodiment, the present disclosure relates to a salt of 2-(5-methoxyisochroman-1-yl)-4,5-dihydro-1H-imidazole (I) with fumaric acid, particularly in crystalline form.

The salt with fumaric acid has been found to exist in two crystalline forms, named herein as crystalline forms 1 and 2.

Fumaric acid salt crystalline form 1 can be prepared, for example, by dissolving compound (I) and fumaric acid, for example, in equivalent molar amounts, in a suitable solvent followed by anti-solvent addition. Suitable solvents include, for example, ethanol, acetone, and 2-propanol; such as 2-propanol. Fumaric acid may be dissolved in 2-propanol before being added to the mixture. Anti-solvent can be added to the solution, for example, in 4 steps, at about room temperature under constant stirring. Suitable anti-solvents include, for example, TBME, water and heptane; such as heptane. After the anti-solvent addition, the mixture is aging at about 5° C. for several hours, for example, about 24-72 hours. The precipitated salt can then be isolated, for example, by filtration or decantation, followed by slow solvent evaporation at about room temperature. The product is then recrystallized by dissolving it in a suitable solvent, for example, ethylacetate at slightly elevated temperature, for example, at about 45-55° C. The recrystallization may be repeated with TBME as a solvent. The obtained solution is allowed to cool, for example, to about room temperature followed by slow solvent evaporation in open vials to yield fumaric acid salt in crystalline form 1.

In one embodiment, the present disclosure relates to crystalline form 1 of the salt of compound (I) with fumaric acid having an X-ray powder diffraction pattern comprising characteristic peaks at about 11.3, 13.2, 15.5, 22.5, 24.2 and 25.3 degrees 2-theta.

In one embodiment, the present disclosure relates to crystalline form 1 of the salt of compound (I) with fumaric acid having an X-ray powder diffraction pattern comprising characteristic peaks at about 7.8, 8.7, 11.3, 13.2, 13.8, 15.5, 16.3, 22.5, 24.2 and 25.3 degrees 2-theta.

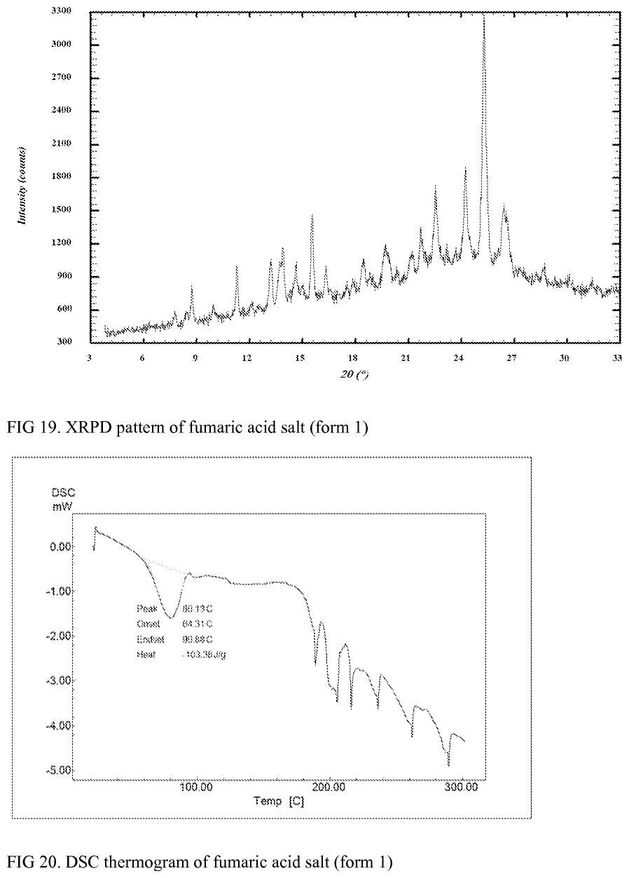

In one embodiment, the crystalline form 1 of the salt of compound (I) with fumaric acid is further characterized by an X-ray powder diffraction pattern as depicted in FIG. 19.

Fumaric acid salt crystalline form 2 can be prepared, for example, by dissolving compound (I) and fumaric acid, for example, in equivalent molar amounts, in a suitable solvent followed by anti-solvent addition. Suitable solvents include, for example, ethanol, acetone, and 2-propanol; such as 2-propanol. Fumaric acid may be dissolved in 2-propanol before being added to the mixture. Anti-solvent can be added to the solution, for example in 4 steps, at about room temperature under constant stirring. Suitable anti-solvents include, for example, TBME, water and heptane; such as heptane. After the anti-solvent addition, the mixture is aging at about 5° C. for several hours, for example, about 24-72 hours. The precipitated salt can then be isolated, for example, by filtration or decantation, followed by slow solvent evaporation at about room temperature. The product is then recrystallized by dissolving it in a suitable solvent, for example, TBME at slightly elevated temperature, for example, at about 45-55° C. The obtained solution is allowed to cool, for example, to about room temperature followed by slow solvent evaporation in open vials to yield fumaric acid salt in crystalline form 2.

In one embodiment, the present disclosure relates to crystalline form 2 of the salt of compound (I) with fumaric acid having an X-ray powder diffraction pattern comprising characteristic peaks at about 10.6, 15.9, 18.0, 22.1 and 26.4 degrees 2-theta.

In one embodiment, the present disclosure relates to crystalline form 2 of the salt of compound (I) with fumaric acid having an X-ray powder diffraction pattern comprising characteristic peaks at about 8.9, 10.6, 12.2, 15.9, 17.7, 18.0, 18.8, 22.1, 24.5, 25.7 and 26.4 degrees 2-theta.

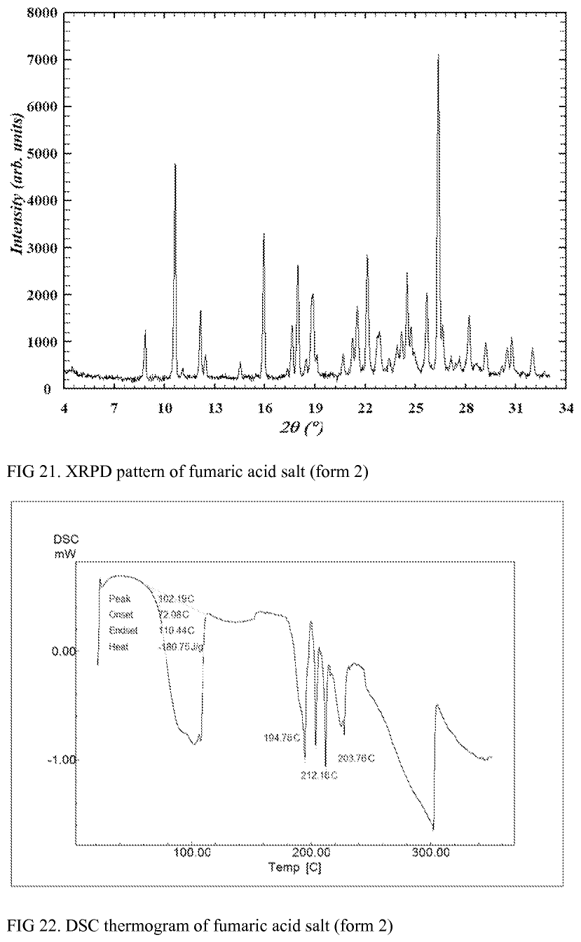

In one embodiment, the crystalline form 2 of the salt of compound (I) with fumaric acid is further characterized by an X-ray powder diffraction pattern as depicted in FIG. 21.

Salt with Glycolic Acid

In one embodiment, the present disclosure relates to a salt of 2-(5-methoxyisochroman-1-yl)-4,5-dihydro-1H-imidazole (I) with glycolic acid, particularly in crystalline form.

The salt with glycolic acid can be prepared, for example, by dissolving compound (I) and glycolic acid, for example, in equivalent molar amounts, in a suitable solvent followed by anti-solvent addition. Suitable solvents include, for example, acetone, ethanol and 2-propanol; such as ethanol. Glycolic acid may be dissolved in ethanol before being added to the mixture. Anti-solvent can be added to the solution, for example, in 4 steps, at about room temperature under constant stirring. Suitable anti-solvents include, for example, water, TBME and heptane; such as TBME. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with glycolic acid appears to precipitate in a single crystalline form, named herein as crystalline form 1. No other crystalline forms have been found for glycolic acid salt.

In one embodiment, the present disclosure relates to crystalline form 1 of the salt of compound (I) with glycolic acid having an X-ray powder diffraction pattern comprising characteristic peaks at about 11.5, 14.5, 17.4, 19.2, 20.6 and 25.3 degrees 2-theta.

In one embodiment, the present disclosure relates to crystalline form 1 of the salt of compound (I) with glycolic acid having an X-ray powder diffraction pattern comprising characteristic peaks at about 9.6, 10.9, 11.5, 14.5, 15.5, 16.8, 17.4, 19.2, 19.5, 20.6, 21.0, 23.0, 23.2 and 25.3 degrees 2-theta.

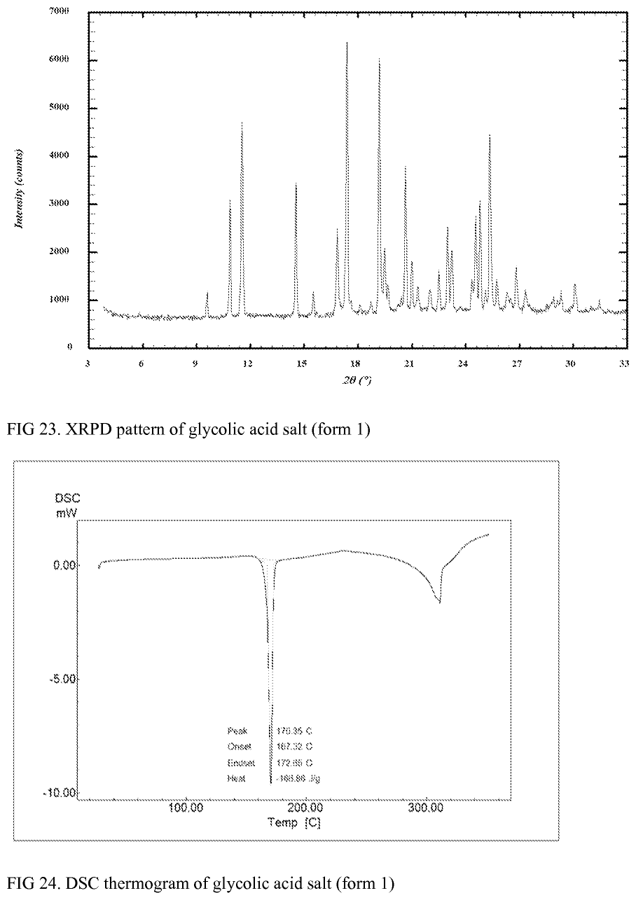

In one embodiment, the crystalline form 1 of the salt of compound (I) with glycolic acid is further characterized by an X-ray powder diffraction pattern as depicted in FIG. 23.

Salt with Glutaric Acid

In one embodiment, the present disclosure relates to a salt of 2-(5-methoxyisochroman-1-yl)-4,5-dihydro-1H-imidazole (I) with glutaric acid, particularly in crystalline form.

The salt with glutaric acid can be prepared, for example, by dissolving compound (I) and glutaric acid, for example, in equivalent molar amounts, in a suitable solvent followed by anti-solvent addition. Suitable solvents include, for example, acetone, ethanol and 2-propanol; such as 2-propanol. Glutaric acid may be dissolved in 2-propanol before being added to the mixture. Anti-solvent can be added to the solution, for example, in 4 steps, at about room temperature under constant stirring. Suitable anti-solvents include, for example, water, TBME and heptane; such as heptane. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with glutaric acid appears to precipitate in a single crystalline form, named herein as crystalline form 1. No other crystalline forms have been found for glutaric acid salt.

In one embodiment, the present disclosure relates to crystalline form 1 of the salt of compound (I) with glutaric acid having an X-ray powder diffraction pattern comprising characteristic peaks at about 12.6, 16.0, 18.8, 19.9 and 21.2 degrees 2-theta.

In one embodiment, the present disclosure relates to crystalline form 1 of the salt of compound (I) with glutaric acid having an X-ray powder diffraction pattern comprising characteristic peaks at about 8.0, 9.8, 10.5, 12.6, 15.5, 16.0, 18.8, 19.9 and 21.2, degrees 2-theta.

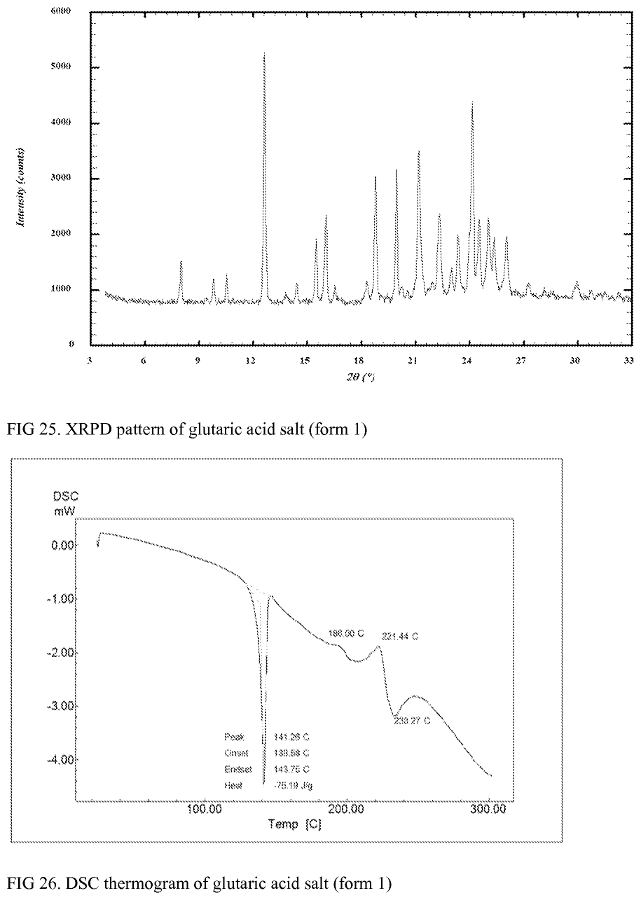

In one embodiment, the crystalline form 1 of the salt of compound (I) with glutaric acid is further characterized by an X-ray powder diffraction pattern as depicted in FIG. 25.

Salt with Adipic Acid

In one embodiment, the present disclosure relates to a salt of 2-(5-methoxyisochroman-1-yl)-4,5-dihydro-1H-imidazole (I) with adipic acid, particularly in crystalline form.

The salt with adipic acid can be prepared, for example, by dissolving compound (I) and adipic acid, for example, in equivalent molar amounts, in a suitable solvent followed by anti-solvent addition. Suitable solvents include, for example, acetone, ethanol and 2-propanol; such as 2-propanol. Adipic acid may be dissolved in 2-propanol before being added to the mixture. Anti-solvent can be added to the solution, for example, in 4 steps, at about room temperature under constant stirring. Suitable anti-solvents include, for example, water, TBME and heptane; such as heptane. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with adipic acid appears to precipitate in a single crystalline form, named herein as crystalline form 1. No other crystalline forms have been found for adipic acid salt.

In one embodiment, the present disclosure relates to crystalline form 1 of the salt of compound (I) with adipic acid having an X-ray powder diffraction pattern comprising characteristic peaks at about 10.5, 12.1, 15.1, 18.3, 20.0 and 21.0 degrees 2-theta.

In one embodiment, the present disclosure relates to crystalline form 1 of the salt of compound (I) with adipic acid having an X-ray powder diffraction pattern comprising characteristic peaks at about 7.7, 10.5, 12.1, 12.4, 14.4, 15.1, 15.4, 17.8, 18.3, 19.6, 20.0 and 21.0 degrees 2-theta.

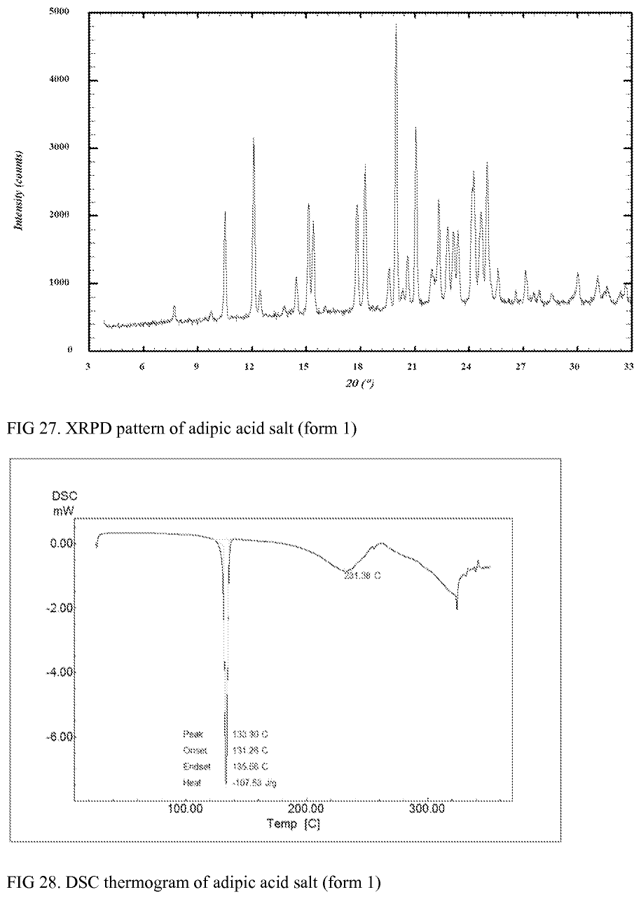

In one embodiment, the crystalline form 1 of the salt of compound (I) with adipic acid is further characterized by an X-ray powder diffraction pattern as depicted in FIG. 27.

Salt with Hydrobromic Acid

In one embodiment, the present disclosure relates to a salt of 2-(5-methoxyisochroman-1-yl)-4,5-dihydro-1H-imidazole (I) with hydrobromic acid, particularly in crystalline form.

The salt with hydrobromic acid can be prepared, for example, by dissolving compound (I), in a suitable solvent, adding hydrobromic acid followed by anti-solvent addition. Suitable solvents include, for example, acetone, ethanol and 2-propanol; such as 2-propanol. Anti-solvent can be added to the solution, for example, in 4 steps, at about room temperature under constant stirring. Suitable anti-solvents include, for example, water, TBME and heptane; such as heptane. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

Alternatively, the salt with hydrobromic acid can be prepared by dissolving compound (I) and hydrobromic acid, for example, in equivalent molar amounts, in a suitable solvent at room or elevated temperature, cooling the mixture, and isolating the crystalline product. Suitable solvents include, for example, ethanol and acetone; such as acetone. The mixture can be heated, for example, to the temperature about 50-70° C., suitably to about 60° C. The obtained solution is stirred and allowed to cool, for example, to about room temperature over several hours, for example, about 2 hours. The mixture can be further cooled to about 5° C. followed by aging at about 5° C. for several hours, for example, about 24-72 hours. The precipitated crystalline salt can then be isolated, for example, by centrifugation and decantation, followed by slow solvent evaporation at about room temperature in open vials.

The salt of compound (I) with hydrobromic acid appears to precipitate in a single crystalline form, named herein as crystalline form 1. No other crystalline forms have been found for hydrobromic acid salt.

In one embodiment, the present disclosure relates to crystalline form 1 of the salt of compound (I) with hydrobromic acid having an X-ray powder diffraction pattern comprising characteristic peaks at about 11.3, 16.9, 20.0, 22.7 and 24.3 degrees 2-theta.

In one embodiment, the present disclosure relates to crystalline form 1 of the salt of compound (I) with hydrobromic acid having an X-ray powder diffraction pattern comprising characteristic peaks at about 5.7, 11.3, 12.5, 13.3, 16.6, 16.9, 17.7, 20.0. 21.9, 22.7 and 24.3 degrees 2-theta.

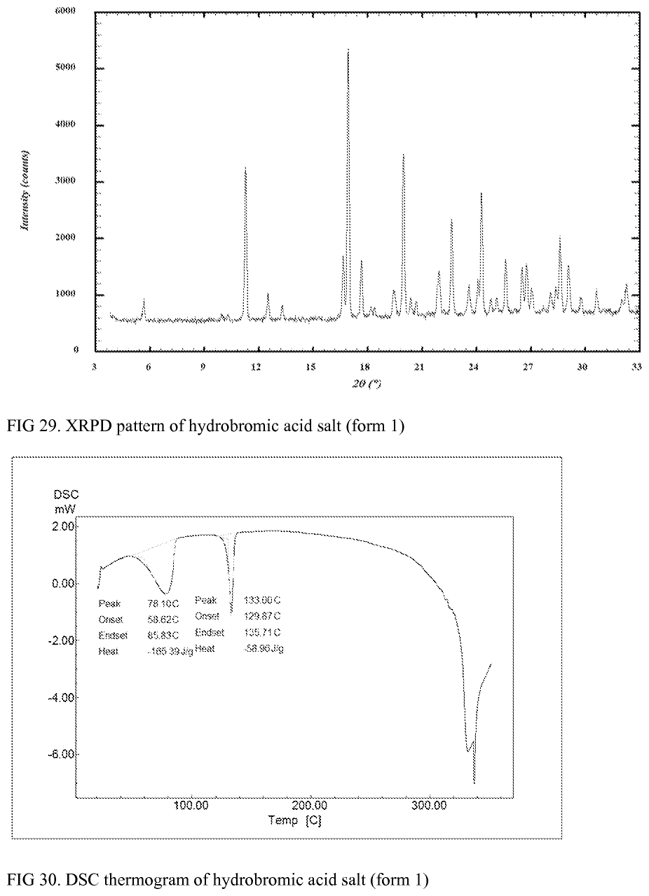

In one embodiment, the crystalline form 1 of the salt of compound (I) with hydrobromic acid is further characterized by an X-ray powder diffraction pattern as depicted in FIG. 29.

Salt with Mucic Acid

In one embodiment, the present disclosure relates to a salt of 2-(5-methoxyisochroman-1-yl)-4,5-dihydro-1H-imidazole (I) with mucic acid, particularly in crystalline form.

The salt with mucic acid can be prepared, for example, by dissolving compound (I) and mucic acid, for example, in equivalent molar amounts, in a suitable solvent followed by anti-solvent addition. Suitable solvents include, for example, acetone, ethanol and 2-propanol; such as ethanol. Mucic acid may be dissolved in dimethyl sulfoxide (DMSO) before being added to the mixture. Anti-solvent can be added to the solution, for example, in 4 steps, at about room temperature under constant stirring. Suitable anti-solvents include, for example, water, TBME and heptane; such as TBME. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials.

The salt of compound (I) with mucic acid appears to precipitate in a single crystalline form, named herein as crystalline form 1. No other crystalline forms have been found for mucic acid salt.

In one embodiment, the present disclosure relates to crystalline form 1 of the salt of compound (I) with mucic acid having an X-ray powder diffraction pattern comprising characteristic peaks at about 5.7, 11.9, 13.9, 17.0, 19.5 and 23.5 degrees 2-theta.

In one embodiment, the present disclosure relates to crystalline form 1 of the salt of compound (I) with mucic acid having an X-ray powder diffraction pattern comprising characteristic peaks at about 5.7, 11.9, 13.9, 17.0, 17.6, 19.3, 19.5, 20.7 and 23.5 degrees 2-theta.

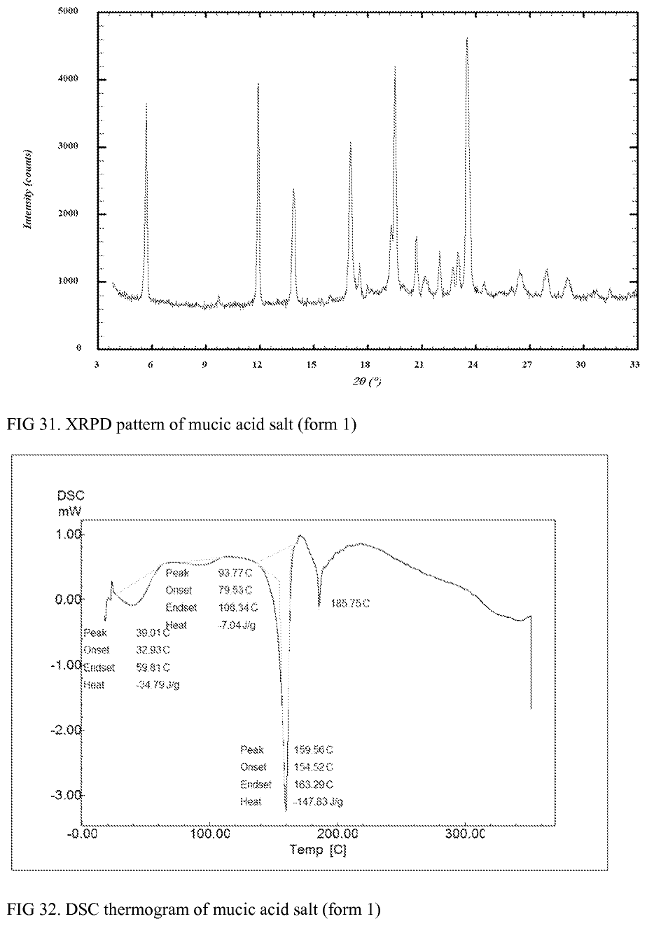

In one embodiment, the crystalline form 1 of the salt of compound (I) with mucic acid is further characterized by an X-ray powder diffraction pattern as depicted in FIG. 31.

Salt with 1-Hydroxy-2-Naphthoic Acid (Xinafoic Acid)

In one embodiment, the present disclosure relates to a salt of 2-(5-methoxyisochroman-1-yl)-4,5-dihydro-1H-imidazole (I) with 1-hydroxy-2-naphthoic acid, particularly in crystalline form.

The salt with 1-hydroxy-2-naphthoic acid can be prepared, for example, by dissolving compound (I) and 1-hydroxy-2-naphthoic acid, for example, in equivalent molar amounts, in a suitable solvent followed by anti-solvent addition. Suitable solvents include, for example, acetone, ethanol and 2-propanol; such as 2-propanol. 1-Hydroxy-2-naphthoic acid may be dissolved in 2-propanol before being added to the mixture. Anti-solvent can be added to the solution, for example, in 4 steps, at about room temperature under constant stirring. Suitable anti-solvents include, for example, water, TBME and heptane; such as heptane. After the anti-solvent addition, the mixture is aging at about 5° C. for several hours, for example, about 24-72 hours. The precipitated crystalline salt can then be isolated, for example, by filtration or decantation, followed by slow solvent evaporation at about room temperature in open vials and optionally, further drying under vacuum.

The salt of compound (I) with 1-hydroxy-2-naphthoic acid appears to precipitate in a single crystalline form, named herein as crystalline form 1. No other crystalline forms have been found for 1-hydroxy-2-naphthoic acid salt.

In one embodiment, the present disclosure relates to crystalline form 1 of the salt of compound (I) with 1-hydroxy-2-naphthoic acid having an X-ray powder diffraction pattern comprising characteristic peaks at about 10.6, 12.5, 15.3, 18.2 and 26.1 degrees 2-theta.

In one embodiment, the present disclosure relates to crystalline form 1 of the salt of compound (I) with 1-hydroxy-2-naphthoic acid having an X-ray powder diffraction pattern comprising characteristic peaks at about 5.5, 6.5, 10.6, 12.5, 15.3, 15.7, 17.0, 17.5, 18.2, 21.3, 21.5 and 26.1 degrees 2-theta.

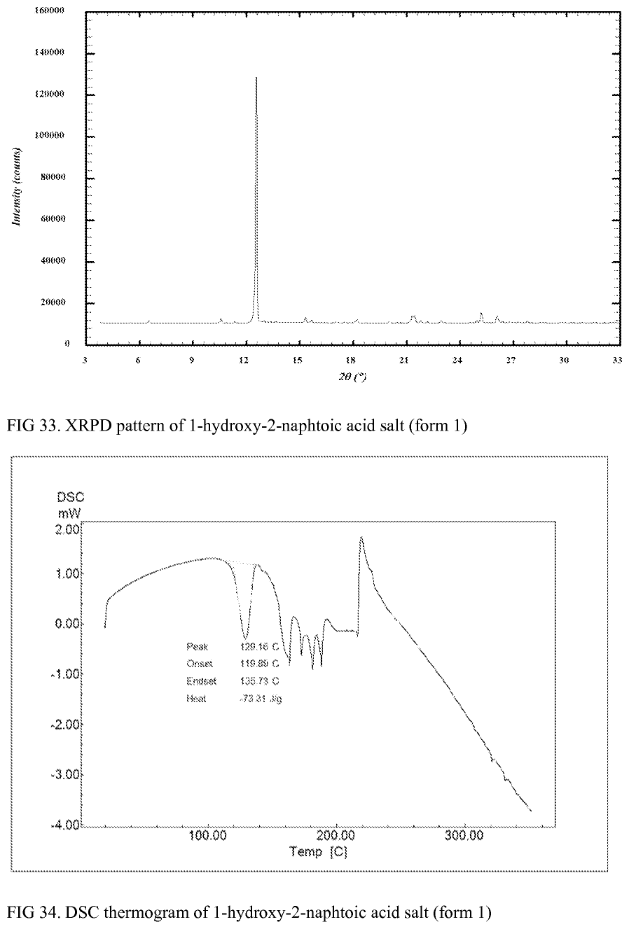

In one embodiment, the crystalline form 1 of the salt of compound (I) with 1-hydroxy-2-naphthoic acid is further characterized by an X-ray powder diffraction pattern as depicted in FIG. 33.

Salt with Hydrochloric Acid

In one embodiment, the present disclosure relates to a salt of 2-(5-methoxyisochroman-1-yl)-4,5-dihydro-1H-imidazole (I) with hydrochloric acid, particularly in crystalline form.

The salt with hydrochloric acid can be prepared, for example, by dissolving compound (I) and hydrochloric acid, for example, in equivalent molar amounts, in a suitable solvent at room or elevated temperature, cooling the mixture, and isolating the crystalline product. Suitable solvents include, for example, ethanol and acetone; such as ethanol. The mixture can be heated, for example, to the temperature about 50-70° C., suitably to about 60° C. The obtained solution is stirred and allowed to cool, for example, to about room temperature over several hours, for example, 2 hours. The mixture can be further cooled to about 5° C. followed by aging at about 5° C. for several hours, for example, about 24-72 hours. The precipitated crystalline salt can then be isolated, for example, by centrifugation and decantation, followed by slow solvent evaporation at about room temperature in open vials.

The salt of compound (I) with hydrochloric acid appears to precipitate in a single crystalline form, named herein as crystalline form 1. No other crystalline forms have been found for hydrochloric acid salt.

In one embodiment, the present disclosure relates to crystalline form 1 of the salt of compound (I) with hydrochloric acid having an X-ray powder diffraction pattern comprising characteristic peaks at about 10.7, 11.0, 12.6, 16.4 and 22.4 degrees 2-theta.

In one embodiment, the present disclosure relates to crystalline form 1 of the salt of compound (I) with hydrochloric acid having an X-ray powder diffraction pattern comprising characteristic peaks at about 5.5, 10.7, 11.0, 12.6, 13.5, 14.1, 16.4, 22.0, 22.4, 24.2 and 25.2 degrees 2-theta.

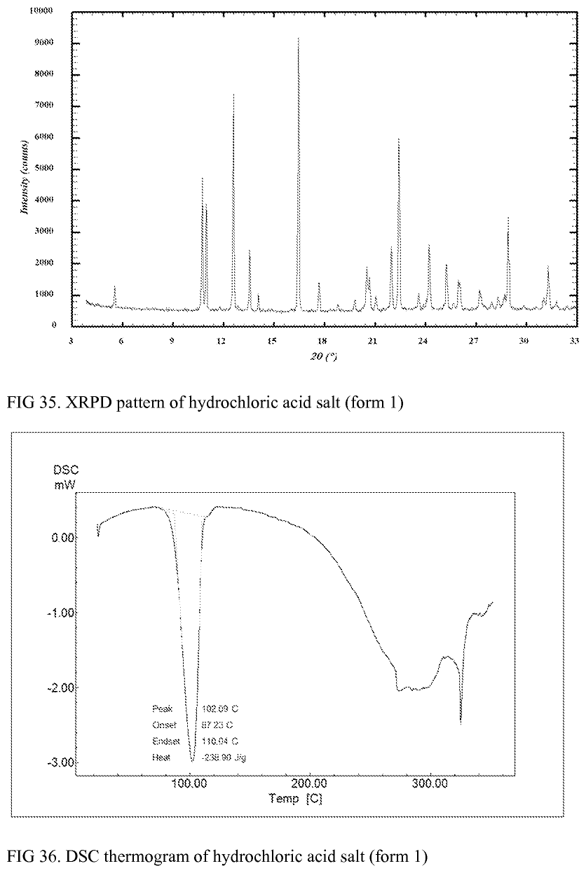

In one embodiment, the crystalline form 1 of the salt of compound (I) with hydrochloric acid is further characterized by an X-ray powder diffraction pattern as depicted in FIG. 35.

The above XRPD peak positions refer to values, when measured using CuKα radiation (λ=1.5418 Å). It is recognized by the skilled person that the X-ray powder diffraction pattern peak positions referred to herein can be subject to variations of ±0.2 degrees 2-theta according to various factors such as temperature, sample handling and instrumentation used.

In one embodiment, the present disclosure relates to substantially pure crystalline forms of the salts of compound (I), as disclosed above, wherein at least 90%, preferably at least 95%, more preferably at least 98%, per weight of the salt of compound (I) is present in said crystalline form.

The above crystalline salts of compound (I) can be formulated into pharmaceutical dosage forms such as tablets, capsules, granules, powders, suspensions or solutions together with one or more excipients which are known in the art.

Thus, in one embodiment, the present disclosure relates to a pharmaceutical composition comprising any of the above salts of compound (I) or crystalline forms thereof together with one or more excipients. In one embodiment, the present disclosure relates to such compositions for use in the treatment or prevention of a disorder, condition or disease where an alpha2A agonist is indicated to be useful, for example, anxiety or condition wherein sedation or analgesia is needed.

In one embodiment, the present disclosure relates to the use of any of the above salts of compound (I) as an active ingredient in the manufacture of a medicament for the treatment or prevention of a disorder, condition or disease where an alpha2A agonist is indicated to be useful, for example, anxiety or condition wherein sedation or analgesia is needed.

In one embodiment, the present disclosure relates to a method for the treatment of a disorder, condition or disease where an alpha2A agonist in indicated to be useful, for example, anxiety or condition wherein sedation or analgesia is needed, which method comprises administering to a subject in need of such treatment a therapeutically effective amount of any of the above salts of compound (I) or a crystalline form thereof.

The present disclosure is further illustrated by the following non-limiting examples.

The salt forms of compound (I) are characterized using the following procedures.

XRPD measurements were performed with the X-ray powder diffractometer Bruker D8 Advance at room temperature, using a copper filled X-ray tube (40 kV×40 mA) as the X-ray source, CuKα (λ=1.5418 Å), a 0.6 mm divergence slit, 2.5° Soller slits on both the primary and secondary beams and the 1-dimensional LynxEye detector (with aperture angle of 2.91593°). Data collection was done in the range of 3.8-33° 2θ with 0.02° increments, at a scan speed of 0.3°/s.

Differential scanning calorimetry (DSC) was performed with a Shimadzu DSC-60 calorimeter in the range of 20-300° C. with a heating rate of 10° C. min-1 in crimped aluminum sample cell with nitrogen flow of 60 ml/min.

The purity analyses were performed using Agilent UHPLC equipment with PDA/DAD detector at 210 nm wavelength and reverse phase liquid chromatography column Acquity UPLC HSS T3, 100×3.0 mm, 1.8 μm (Waters). The chromatography was performed with gradient run using acetonitrile and phosphate buffer (pH 3.0) as eluents.

### Example 1: Naphtalene-1,5-Disulfonic Acid Salt Crystalline Form 1

200 mg of 2-(5-methoxyisochroman-1-yl)-4,5-dihydro-1H-imidazole free base (I) was dissolved in 12 ml of acetone, followed by vortexing until complete dissolution. 273.5 mg of naphtalene-1,5-disulfonic acid was pre-dissolved in 0.5 ml of water and 0.5 ml of acetone and was added over the free base solution to a 1:1 molar ratio of free base:acid.

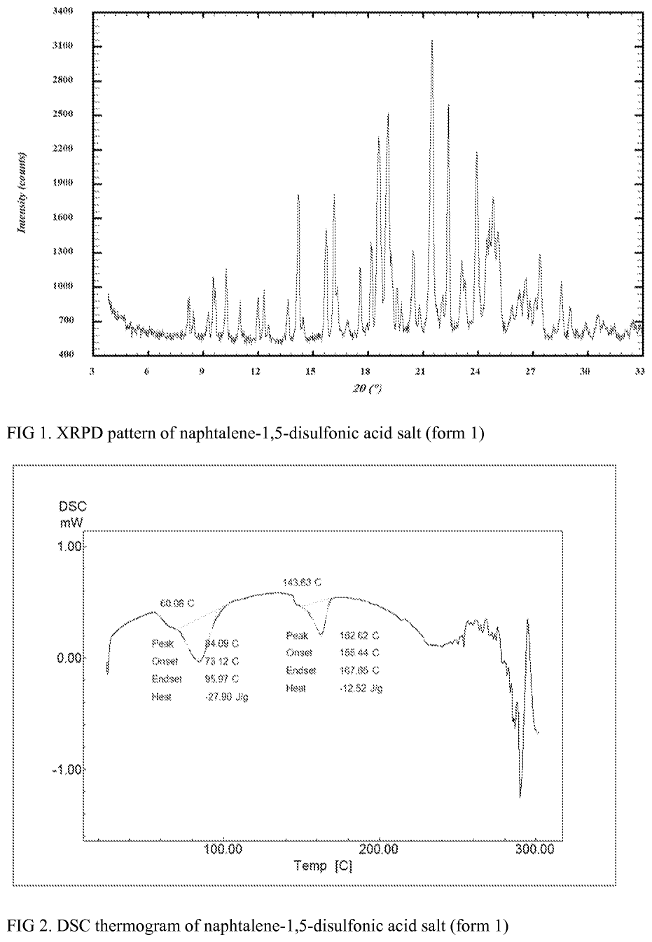

Subsequently, the anti-solvent (water) was added in 4 steps, at room temperature (RT) and under constant stirring. The total anti-solvent volume was 0.5, 1, 2 and 4 times the free base solution volume. A clear solution was obtained. Next, the sample was aged at 5° C. for 72 h followed by solvent removal at RT, in open vial, for 2 days. For complete solvent removal, the sample was vacuum-dried (200 mbar, 40° C., 3 days). Next, the sample was recrystallized by adding 4 ml ethylacetate, heating at 50° C. for 30 min, followed by slow solvent evaporation in open vial. The white-off powder was analyzed by XRPD. To confirm the salt form stability after complete drying, the sample was vacuum-dried (150 mbar, 30° C., 3 h) and re-analyzed by XRPD and was found to be a crystalline form 1 (table 1). The X-ray powder diffraction pattern of form 1 is depicted in FIG. 1 and the DSC thermogram in FIG. 2. DSC analysis shows two endotherms with onset temperatures approximately 73° C. and 155° C.

TABLE 1
X-ray powder reflections (up to 33° 2θ) and intensities
(normalized) of naphtalene-1,5-disulfonic acid salt crystalline
form 1. The value 2θ [°] represents the diffraction
angle in degrees and the value d [Å] represents the
specified distances in Å between the lattice planes.

| 2θ [°] | d [Å] | I / Iₒ [%] |
|---|---|---|
| 8.20 | 10.78 | 27 |
| 8.46 | 10.44 | 26 |
| 9.27 | 9.54 | 26 |
| 9.57 | 9.23 | 30 |
| 10.24 | 8.63 | 37 |
| 11.00 | 8.04 | 28 |
| 11.99 | 7.38 | 29 |
| 12.30 | 7.19 | 30 |
| 12.55 | 7.05 | 23 |
| 13.61 | 6.50 | 29 |
| 14.18 | 6.24 | 60 |
| 14.44 | 6.13 | 24 |
| 15.69 | 5.64 | 48 |
| 16.16 | 5.48 | 55 |
| 16.34 | 5.42 | 33 |
| 16.89 | 5.24 | 24 |
| 17.57 | 5.04 | 38 |
| 18.17 | 4.88 | 46 |
| 18.58 | 4.77 | 76 |
| 19.06 | 4.65 | 79 |
| 19.59 | 4.53 | 33 |
| 19.81 | 4.48 | 28 |
| 20.45 | 4.34 | 43 |
| 20.80 | 4.27 | 28 |
| 21.46 | 4.14 | 100 |
| 22.04 | 4.03 | 31 |
| 22.36 | 3.97 | 85 |
| 23.15 | 3.84 | 37 |
| 23.92 | 3.72 | 72 |
| 24.49 | 3.63 | 47 |
| 24.80 | 3.59 | 57 |
| 25.11 | 3.54 | 49 |
| 25.83 | 3.45 | 28 |
| 26.26 | 3.39 | 32 |
| 26.57 | 3.35 | 35 |
| 26.88 | 3.31 | 29 |
| 27.16 | 3.28 | 30 |
| 27.36 | 3.26 | 41 |
| 28.12 | 3.17 | 22 |
| 28.53 | 3.13 | 34 |
| 29.03 | 3.07 | 26 |

### Example 2: Ethane-1,2-Disulfonic Acid Salt Crystalline Form 1

200.2 mg of 2-(5-methoxyisochroman-1-yl)-4,5-dihydro-1H-imidazole free base (I) was dissolved in 10 ml of 2-propanol, followed by vortexing until complete dissolution. 201.2 mg of ethane-1,2-disulfonic acid hydrate was pre-dissolved in 1 ml of water and 1 ml of 2-propanol with heating at 50° C. for 5 min. The resulting solution was added over the free base solution to a 1:1 molar ratio of free base:acid. A clear solution was obtained. Subsequently, the anti-solvent (heptane) was added in 2 steps, at RT and under constant stirring. The total anti-solvent volume was 0.5 and 1 time the FB solution volume. Precipitation was observed. Next, the sample was aged at 5° C. for 72 h. After completing the ageing time, the sample was decanted and the precipitate was air-dried at RT, in open vial, for 1 day. The resulting white powder was analyzed by XRPD. To confirm the salt form stability after complete drying, the sample was vacuum-dried (150 mbar, 30° C., 3 h) and re-analyzed by XRPD and was found to be a crystalline form 1 (table 2). The X-ray powder diffraction pattern of form 1 is depicted in FIG. 3 and the DSC thermogram in FIG. 4. DSC analysis shows an endotherm with an onset temperature approximately 254° C.

TABLE 2
X-ray powder reflections (up to 33° 2θ) and intensities (normalized)
of ethane-1,2-disulfonic acid salt crystalline form 1. The value 2θ
[°] represents the diffraction angle in degrees and the value
d [Å] represents the specified distances in Å between the lattice planes.

| 2θ [°] | d [Å] | I / Iₒ [%] |
|---|---|---|
| 9.90 | 8.93 | 8 |
| 12.13 | 7.29 | 6 |
| 13.51 | 6.55 | 100 |
| 14.50 | 6.10 | 14 |
| 15.37 | 5.76 | 3 |
| 16.02 | 5.53 | 22 |
| 19.88 | 4.46 | 17 |
| 20.05 | 4.43 | 19 |
| 20.32 | 4.37 | 42 |
| 21.24 | 4.18 | 2 |
| 21.71 | 4.09 | 14 |
| 22.77 | 3.90 | 83 |
| 23.28 | 3.82 | 4 |
| 24.55 | 3.62 | 7 |
| 25.02 | 3.56 | 14 |
| 27.88 | 3.20 | 22 |
| 28.24 | 3.16 | 2 |
| 28.76 | 3.10 | 3 |
| 28.90 | 3.09 | 3 |
| 29.13 | 3.06 | 6 |
| 30.08 | 2.97 | 1 |
| 30.67 | 2.91 | 4 |
| 32.32 | 2.77 | 4 |

### Example 3: Oxalic Acid Salt Crystalline Form 1

200 mg of 2-(5-methoxyisochroman-1-yl)-4,5-dihydro-1H-imidazole free base (I) was dissolved in 12 ml of acetone. 87.2 mg of oxalic acid was pre-dissolved in 1 ml acetone and was added over the free base solution to a 1:1 molar ratio of free base:acid. Precipitation was observed. The resulting suspension was heated at 60° C. for 30 min followed by cooling at RT during 2 h and ageing at 5° C. for 24 h. After completing the ageing time, the sample was centrifuged, decanted and the precipitate was air-dried at RT, in open vial, for 3 days. The white powder was analyzed by XRPD. To confirm the salt form stability after complete drying, the sample was vacuum-dried (150 mbar, 30° C., 3 h) and re-analyzed by XRPD and was found to be a crystalline form 1 (table 3). The X-ray powder diffraction pattern of form 1 is depicted in FIG. 5 and the DSC thermogram in FIG. 6. DSC analysis shows an endotherm with an onset temperature approximately 217° C.

TABLE 3
X-ray powder reflections (up to 33° 2θ) and intensities
(normalized) of oxalic acid salt crystalline form 1. The value
2θ [°] represents the diffraction angle in degrees
and the value d [Å] represents the specified distances
in Å between the lattice planes.

| 2θ [°] | d [Å] | I / Iₒ [%] |
|---|---|---|
| 5.87 | 15.04 | 3 |
| 10.76 | 8.22 | 10 |
| 11.70 | 7.56 | 13 |
| 12.08 | 7.32 | 10 |
| 14.31 | 6.18 | 17 |
| 16.24 | 5.45 | 9 |
| 16.50 | 5.37 | 15 |
| 16.96 | 5.22 | 22 |
| 17.58 | 5.04 | 100 |
| 18.94 | 4.68 | 12 |
| 19.25 | 4.61 | 6 |
| 19.75 | 4.49 | 34 |
| 21.29 | 4.17 | 6 |
| 22.08 | 4.02 | 12 |
| 22.33 | 3.98 | 36 |
| 23.50 | 3.78 | 46 |
| 24.19 | 3.68 | 8 |
| 24.56 | 3.62 | 7 |
| 25.25 | 3.52 | 4 |
| 26.65 | 3.34 | 7 |
| 29.93 | 2.98 | 4 |
| 31.80 | 2.81 | 2 |
| 32.37 | 2.76 | 4 |

### Example 4: Saccharin Salt Crystalline Form 1

15.2 mg of 2-(5-methoxyisochroman-1-yl)-4,5-dihydro-1H-imidazole free base (I) was dissolved in 0.2 ml of ethanol. 13.6 mg of saccharin was dissolved in 0.4 ml of ethanol at 50° C. and was added over the free base solution to a 1:1 molar ratio of free base:acid. A clear solution was obtained. The solution was heated at 60° C. for 30 min, then cooled at RT during 2 h and aged at 5° C. for 24 h. After completing the ageing time, solvent removal was achieved at RT, in open vial, for 4 days. A clay-like material was obtained. Next, 0.2 ml of ethylacetate was added, the mixture was heated at 50° C. for 30 min, followed by slow solvent evaporation in open vial. A clay-like material was obtained again. Next, 0.2 ml of TBME was added, the mixture was heated at 50° C. for 30 min, followed by slow solvent evaporation at RT in open vial. The off-yellow powder was analyzed by XRPD and was found to be a crystalline form 1 (table 4). The X-ray powder diffraction pattern of form 1 is depicted in FIG. 7 and the DSC thermogram in FIG. 8. DSC analysis shows an endotherm with an onset temperature approximately 133° C.

TABLE 4
X-ray powder reflections (up to 33° 2θ) and intensities
(normalized) of saccharin salt crystalline form 1. The value
2θ [°] represents the diffraction angle in
degrees and the value d [Å] represents the specified
distances in Å between the lattice planes.

| 2θ [°] | d [Å] | I / Iₒ [%] |
|---|---|---|
| 9.39 | 9.41 | 76 |
| 10.75 | 8.22 | 3 |
| 12.40 | 7.13 | 3 |
| 13.50 | 6.55 | 49 |
| 15.06 | 5.88 | 2 |
| 15.55 | 5.69 | 32 |
| 16.04 | 5.52 | 10 |
| 17.11 | 5.18 | 54 |
| 17.76 | 4.99 | 3 |
| 18.24 | 4.86 | 6 |
| 18.80 | 4.72 | 100 |
| 19.11 | 4.64 | 4 |
| 19.50 | 4.55 | 16 |
| 20.12 | 4.41 | 3 |
| 20.52 | 4.32 | 16 |
| 20.90 | 4.25 | 7 |
| 21.47 | 4.13 | 47 |
| 22.57 | 3.94 | 5 |
| 23.00 | 3.86 | 17 |
| 23.67 | 3.76 | 8 |
| 24.34 | 3.65 | 14 |
| 24.90 | 3.57 | 10 |
| 25.30 | 3.52 | 34 |
| 25.78 | 3.45 | 3 |
| 26.29 | 3.39 | 9 |
| 26.60 | 3.35 | 24 |
| 27.16 | 3.28 | 4 |
| 28.25 | 3.16 | 16 |
| 28.94 | 3.08 | 4 |
| 29.36 | 3.04 | 2 |
| 29.92 | 2.98 | 3 |
| 30.82 | 2.90 | 4 |
| 31.33 | 2.85 | 6 |
| 31.88 | 2.81 | 3 |

### Example 5: Saccharin Salt Crystalline Form 2

200.4 mg of 2-(5-methoxyisochroman-1-yl)-4,5-dihydro-1H-imidazole free base (I) was dissolved in 7 ml of ethanol, followed by vortexing until complete dissolution. 177.3 mg of saccharin was pre-dissolved in 5 ml of ethanol by heating at 50° C. for 5 min and was added over the free base solution to a 1:1 molar ratio of free base:acid. A clear solution was obtained. Subsequently, the anti-solvent (TBME) was added in 4 steps, at RT and under constant stirring. The total anti-solvent volume was 0.5, 1, 2 and 4 times the free base solution volume. A clear solution was obtained. Next, the sample was aged at 5° C. for 72 h followed by solvent removal at RT, in open vial, for 3 days. The white powder was analyzed by XRPD. To confirm the salt form stability after complete drying, the sample was vacuum-dried (150 mbar, 30° C., 3 h) and re-analyzed by XRPD and was found to be a crystalline form 2 (table 5). The X-ray powder diffraction pattern of form 2 is depicted in FIG. 9 and the DSC thermogram in FIG. 10. DSC analysis shows an endotherm with an onset temperature approximately 137° C.

TABLE 5
X-ray powder reflections (up to 33° 2θ) and intensities
(normalized) of saccharin salt crystalline form 2. The value
2θ [°] represents the diffraction angle in
degrees and the value d [Å] represents the specified
distances in Å between the lattice planes.

| 2θ [°] | d [Å] | I / Iₒ [%] |
|---|---|---|
| 7.47 | 11.83 | 4 |
| 8.52 | 10.36 | 13 |
| 9.23 | 9.57 | 21 |
| 12.47 | 7.10 | 6 |
| 13.19 | 6.71 | 9 |
| 14.06 | 6.29 | 26 |
| 14.38 | 6.16 | 10 |
| 15.05 | 5.88 | 85 |
| 15.40 | 5.75 | 3 |
| 15.84 | 5.59 | 67 |
| 16.16 | 5.48 | 36 |
| 16.33 | 5.42 | 31 |
| 16.53 | 5.36 | 4 |
| 17.13 | 5.17 | 3 |
| 17.73 | 5.00 | 100 |
| 18.48 | 4.80 | 7 |
| 18.74 | 4.73 | 22 |
| 19.13 | 4.64 | 23 |
| 20.10 | 4.41 | 8 |
| 20.88 | 4.25 | 13 |
| 21.37 | 4.16 | 18 |
| 22.22 | 4.00 | 7 |
| 22.49 | 3.95 | 21 |
| 23.16 | 3.84 | 32 |
| 23.88 | 3.72 | 27 |
| 24.17 | 3.68 | 53 |
| 25.05 | 3.55 | 28 |
| 25.22 | 3.53 | 48 |
| 25.78 | 3.45 | 23 |
| 26.10 | 3.41 | 22 |
| 26.57 | 3.35 | 70 |
| 27.09 | 3.29 | 6 |
| 27.33 | 3.26 | 3 |
| 27.92 | 3.19 | 3 |
| 28.31 | 3.15 | 47 |
| 28.66 | 3.11 | 3 |
| 28.95 | 3.08 | 9 |
| 30.81 | 2.90 | 4 |
| 31.56 | 2.83 | 4 |
| 31.97 | 2.80 | 3 |

### Example 6: Maleic Acid Salt Crystalline Form 1

200 mg of 2-(5-methoxyisochroman-1-yl)-4,5-dihydro-1H-imidazole free base (I) was dissolved in 10 ml of 2-propanol, followed by vortexing until complete dissolution. 112.2 mg of maleic acid was dissolved in 1 ml of 2-propanol and was added over the free base solution to a 1:1 molar ratio of free base:acid. A clear solution was obtained. Subsequently, the anti-solvent (heptane) was added in 4 steps, at RT and under constant stirring. The total anti-solvent volume was 0.5, 1, 2 and 4 times the free base solution volume. Precipitation was observed. Next, the sample was aged at 5° C. for 72 h. After completing the ageing time, the sample was decanted and the precipitate was air-dried at RT, in open vial, for 1 day. The resulting white powder was analyzed by XRPD. To confirm the salt form stability after complete drying, the sample was vacuum-dried (150 mbar, 30° C., 3 h) and re-analyzed by XRPD and was found to be a crystalline form 1 (table 6). The X-ray powder diffraction pattern of form 1 is depicted in FIG. 11 and the DSC thermogram in FIG. 12. DSC analysis shows an endotherm with an onset temperature approximately 121° C.

TABLE 6
X-ray powder reflections (up to 33° 2θ) and intensities
(normalized) of maleic acid salt crystalline form 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 10.21 | 8.66 | 33 |
| 11.59 | 7.63 | 69 |
| 14.32 | 6.18 | 26 |
| 16.80 | 5.27 | 100 |
| 17.07 | 5.19 | 12 |
| 17.39 | 5.10 | 86 |
| 19.30 | 4.59 | 38 |
| 21.02 | 4.22 | 8 |
| 22.03 | 4.03 | 15 |
| 22.28 | 3.99 | 24 |
| 22.43 | 3.96 | 19 |
| 23.26 | 3.82 | 19 |
| 23.93 | 3.72 | 38 |
| 24.10 | 3.69 | 42 |
| 24.78 | 3.59 | 13 |
| 25.55 | 3.48 | 10 |
| 26.84 | 3.32 | 5 |
| 27.92 | 3.19 | 19 |
| 28.12 | 3.17 | 11 |
| 30.11 | 2.97 | 4 |
| 30.84 | 2.90 | 3 |
| 31.26 | 2.86 | 3 |

### Example 7: Malonic Acid Salt Crystalline Form 1

200.1 mg of 2-(5-methoxyisochroman-1-yl)-4,5-dihydro-1H-imidazole free base (I) was dissolved in 7 ml of ethanol, followed by vortexing until complete dissolution. 99.1 mg of malonic acid was pre-dissolved in 1 ml of ethanol and was added over the free base solution to a 1:1 molar ratio of free base:acid. A clear solution was obtained. Subsequently, the anti-solvent (TBME) was added in 4 steps, at RT and under constant stirring; the total anti-solvent volume was 0.5, 1, 2 and 4 times the free base solution volume. Precipitation was observed. Next, the sample was aged at 5° C. for 72 h. After completing the ageing time, the sample was decanted and the precipitate was air-dried at RT, in open vial, for 1 day. The resulting white powder was analyzed by XRPD. To confirm the salt form stability after complete drying, the sample was vacuum-dried (150 mbar, 30° C., 3 h) and re-analyzed by XRPD and was found to be a crystalline form 1 (table 7). The X-ray powder diffraction pattern of form 1 is depicted in FIG. 13 and the DSC thermogram in FIG. 14. DSC analysis shows an endotherm with an onset temperature approximately 145° C.

TABLE 7
X-ray powder reflections (up to 33° 2θ) and intensities
(normalized) of malonic acid salt crystalline form 1. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 10.10 | 8.75 | 46 |
| 10.35 | 8.54 | 39 |
| 10.83 | 8.16 | 42 |
| 13.70 | 6.46 | 15 |
| 14.08 | 6.28 | 80 |
| 16.26 | 5.45 | 100 |
| 17.38 | 5.10 | 92 |
| 18.60 | 4.77 | 8 |
| 19.34 | 4.58 | 7 |
| 19.74 | 4.49 | 9 |
| 20.28 | 4.38 | 3 |
| 20.77 | 4.27 | 7 |
| 21.48 | 4.13 | 13 |
| 21.76 | 4.08 | 36 |
| 22.07 | 4.02 | 19 |
| 22.82 | 3.89 | 5 |
| 23.25 | 3.82 | 4 |
| 23.65 | 3.76 | 21 |
| 24.02 | 3.70 | 74 |
| 24.18 | 3.68 | 37 |
| 24.65 | 3.61 | 41 |
| 25.02 | 3.56 | 7 |
| 25.34 | 3.51 | 11 |
| 26.26 | 3.39 | 9 |
| 26.97 | 3.30 | 30 |
| 27.41 | 3.25 | 4 |
| 28.09 | 3.17 | 10 |
| 28.71 | 3.11 | 8 |
| 29.15 | 3.06 | 5 |
| 30.70 | 2.91 | 3 |
| 31.32 | 2.85 | 7 |
| 32.25 | 2.77 | 6 |

### Example 8: L-Tartaric Acid Salt Crystalline Form 1

200.2 mg of 2-(5-methoxyisochroman-1-yl)-4,5-dihydro-1H-imidazole free base (I) was dissolved in 7 ml of ethanol, followed by vortexing until complete dissolution. 143.6 mg of L-tartaric acid was pre-dissolved in 1 ml of ethanol by heating at 50° C. for 5 min and was added over the free base solution to a 1:1 molar ratio of free base:acid. A clear solution was obtained. Subsequently, the anti-solvent (TBME) was added in 4 steps, at RT and under constant stirring. The total anti-solvent volume was 0.5, 1, 2 and 4 times the free base solution volume. A precipitation was observed. Next, the sample was aged at 5° C. for 72 h. After completing the ageing time, the sample was decanted and the sticky precipitate was air-dried at RT, in open vial, for 1 day. Next, the sample was recrystallized by adding 4 ml of ethylacetate, heating at 50° C. for 30 min, followed by slow evaporation at RT in open vial and further drying under vacuum (150 mbar, 30° C., 3 h). Finally, the white powder was exposed to 40° C. and 75% relative humidity for 3 weeks and then analyzed by XRPD, and was found to be a crystalline form 1 (table 8). The X-ray powder diffraction pattern of form 1 is depicted in FIG. 15 and the DSC thermogram in FIG. 16. DSC analysis shows two endotherms with onset temperatures approximately 57° C. and 152° C.

TABLE 8
X-ray powder reflections (up to 33° 2θ) and intensities (normalized)
of L-tartaric acid salt crystalline form 1. The value 2θ [°]
represents the diffraction angle in degrees and the value d [Å]
represents the specified distances in Å between the lattice planes.

| 2θ [°] | d [Å] | I/Iₒ [%] |
|---|---|---|
| 7.16 | 12.34 | 17 |
| 10.10 | 8.75 | 17 |
| 10.85 | 8.15 | 8 |
| 11.75 | 7.53 | 100 |
| 12.06 | 7.33 | 9 |
| 12.52 | 7.06 | 46 |
| 12.86 | 6.88 | 59 |
| 13.42 | 6.59 | 82 |
| 13.73 | 6.44 | 37 |
| 13.95 | 6.34 | 78 |
| 14.32 | 6.18 | 8 |
| 15.44 | 5.73 | 16 |
| 15.97 | 5.55 | 16 |
| 16.77 | 5.28 | 10 |
| 18.17 | 4.88 | 23 |
| 18.80 | 4.72 | 43 |
| 19.31 | 4.59 | 39 |
| 20.00 | 4.44 | 38 |
| 20.28 | 4.38 | 37 |
| 20.78 | 4.27 | 43 |
| 21.02 | 4.22 | 20 |
| 21.33 | 4.16 | 25 |
| 21.72 | 4.09 | 26 |
| 22.51 | 3.95 | 38 |
| 23.04 | 3.86 | 27 |
| 23.43 | 3.79 | 34 |
| 23.96 | 3.71 | 59 |
| 25.28 | 3.52 | 24 |
| 26.08 | 3.41 | 23 |
| 27.01 | 3.30 | 18 |
| 28.12 | 3.17 | 9 |
| 29.73 | 3.00 | 9 |
| 31.58 | 2.83 | 8 |

### Example 9: L-Tartaric Acid Salt Crystalline Form 2

200.2 mg of 2-(5-methoxyisochroman-1-yl)-4,5-dihydro-1H-imidazole free base (I) was dissolved in 7 ml of ethanol, followed by vortexing until complete dissolution. 143.6 mg of L-tartaric acid was pre-dissolved in 1 ml of ethanol by heating at 50° C. for 5 min and was added over the free base solution to a 1:1 molar ratio of free base:acid. A clear solution was obtained. Subsequently, the anti-solvent (TBME) was added in 4 steps, at RT and under constant stirring. The total anti-solvent volume was 0.5, 1, 2 and 4 times the free base solution volume. Precipitation was observed. Next, the sample was aged at 5° C. for 72 h. After completing the ageing time, the sample was decanted and the sticky precipitate was air-dried at RT, in open vial, for 1 day. Next, the sample was recrystallized by adding 4 ml of ethylacetate, heating at 50° C. for 30 min, followed by slow evaporation at RT in open vial and further drying under vacuum (150 mbar, 30° C., 3 h). Finally, the white powder was exposed to 40° C. and 75% relative humidity for 3 weeks and then analyzed by XRPD, and was found to be a crystalline form 2 (table 9). The X-ray powder diffraction pattern of form 2 is depicted in FIG. 17 and the DSC thermogram in FIG. 18. DSC analysis shows two endotherms with onset temperatures approximately 148° C. and 158° C.

TABLE 9
X-ray powder reflections (up to 33° 2θ) and intensities (normalized)
of L-tartaric acid salt crystalline form 2. The value 2θ [°]
represents the diffraction angle in degrees and the value d [Å]
represents the specified distances in Å between the lattice planes.

| 2θ [°] | d [Å] | I/Iₒ [%] |
|---|---|---|
| 7.22 | 12.24 | 78 |
| 11.33 | 7.80 | 31 |
| 11.75 | 7.53 | 10 |
| 12.04 | 7.35 | 77 |
| 12.56 | 7.04 | 27 |
| 13.54 | 6.53 | 93 |
| 13.95 | 6.34 | 10 |
| 14.41 | 6.14 | 5 |
| 14.96 | 5.91 | 54 |
| 15.16 | 5.84 | 34 |
| 15.64 | 5.66 | 23 |
| 15.79 | 5.61 | 42 |
| 16.42 | 5.39 | 31 |
| 17.05 | 5.20 | 38 |
| 18.10 | 4.90 | 16 |
| 18.45 | 4.81 | 29 |
| 19.07 | 4.65 | 76 |
| 20.06 | 4.42 | 8 |
| 20.83 | 4.26 | 10 |
| 21.14 | 4.20 | 95 |
| 21.48 | 4.13 | 61 |
| 21.67 | 4.10 | 61 |
| 22.35 | 3.97 | 24 |
| 22.73 | 3.91 | 76 |
| 23.00 | 3.86 | 44 |
| 23.35 | 3.81 | 12 |
| 23.90 | 3.72 | 17 |
| 24.15 | 3.68 | 18 |
| 24.62 | 3.61 | 100 |
| 25.21 | 3.53 | 96 |
| 25.49 | 3.49 | 46 |
| 27.38 | 3.26 | 11 |
| 27.78 | 3.21 | 15 |
| 29.39 | 3.04 | 10 |
| 30.11 | 2.97 | 12 |
| 30.39 | 2.94 | 16 |
| 31.75 | 2.82 | 15 |
| 32.70 | 2.74 | 7 |

### Example 10: Fumaric Acid Salt Crystalline Form 1

15 mg of 2-(5-methoxyisochroman-1-yl)-4,5-dihydro-1H-imidazole free base (I) was dissolved in 0.8 ml of 2-propanol, followed by vortexing until complete dissolution. 8.6 mg of fumaric acid was pre-dissolved in 0.2 ml of 2-propanol by heating at 50° C. for 5 min and was added over the free base solution to a 1:1 molar ratio of free base:acid. A clear solution was obtained. Subsequently, the anti-solvent was added in 4 steps, at RT and under constant stirring. The total anti-solvent volume was 0.5, 1, 2 and 4 times the free base solution volume. Light precipitate was observed. Next, the sample was aged at 5° C. for 24 h following by solvent removal at RT, in open vial, for 2 days. A clay-like material was obtained. Next, the sample was recrystallized by adding 0.3 ml of ethylacetate, heating at 50° C. for 30 min and solvent evaporation at RT in open vial. Sticky material was obtained. A second attempt of recrystallization was performed by adding 0.3 ml of TBME, heating at 50° C. for 30 min, followed by slow evaporation at RT in open vial. The white powder was analyzed by XRPD and was found to be a crystalline form 1 (table 10). The X-ray powder diffraction pattern of form 1 is depicted in FIG. 19 and the DSC thermogram in FIG. 20. DSC analysis shows an endotherm with an onset temperature approximately 64° C.

TABLE 10
X-ray powder reflections (up to 33° 2θ) and intensities
(normalized) of fumaric acid salt crystalline form 1. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 7.75 | 11.40 | 6 |
| 8.68 | 10.18 | 16 |
| 9.92 | 8.91 | 6 |
| 11.27 | 7.84 | 20 |
| 13.19 | 6.70 | 20 |
| 13.81 | 6.41 | 17 |
| 14.61 | 6.06 | 15 |
| 15.53 | 5.70 | 34 |
| 16.31 | 5.43 | 12 |
| 18.45 | 4.81 | 12 |
| 19.66 | 4.51 | 14 |
| 21.21 | 4.19 | 8 |
| 21.70 | 4.09 | 18 |
| 22.54 | 3.94 | 33 |
| 24.23 | 3.67 | 37 |
| 25.29 | 3.52 | 100 |
| 26.42 | 3.37 | 24 |

### Example 11: Fumaric Acid Salt Crystalline Form 2

200.1 mg of 2-(5-methoxyisochroman-1-yl)-4,5-dihydro-1H-imidazole free base (I) was dissolved in 10 ml of 2-propanol, followed by vortexing until complete dissolution. 111.2 mg of fumaric acid was pre-dissolved in 2 ml of 2-propanol by heating at 50° C. for 5 min and was added over the free base solution to a 1:1 molar ratio of free base:acid. A clear solution was obtained. Subsequently, the anti-solvent (heptane) was added in 4 steps, at RT and under constant stirring. The total anti-solvent volume was 0.5, 1, 2 and 4 times the free base solution volume. Sticky precipitate was observed. Next, the sample was aged at 5° C. for 72 h followed by solvent removal at RT, in open vial, for 4 days. A clay-like material was obtained. The sample was recrystallized by adding 4 ml TBME, heating at 50° C. for 30 min, followed by slow evaporation at RT in open vial. The resulting white powder was analyzed by XRPD. To confirm the salt form stability after complete drying, the sample was vacuum-dried (150 mbar, 30° C., 3 h) and re-analyzed by XRPD and was found to be a crystalline form 2 (table 11). The X-ray powder diffraction pattern of form 1 is depicted in FIG. 21 and the DSC thermogram in FIG. 22. DSC analysis shows an endotherm with an onset temperature approximately 72° C.

TABLE 11
X-ray powder reflections (up to 33° 2θ) and intensities
(normalized) of fumaric acid salt crystalline form 2. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 8.85 | 9.99 | 15 |
| 10.63 | 8.31 | 68 |
| 11.08 | 7.98 | 3 |
| 12.15 | 7.28 | 21 |
| 12.45 | 7.10 | 7 |
| 14.54 | 6.09 | 5 |
| 15.93 | 5.56 | 45 |
| 17.33 | 5.11 | 3 |
| 17.67 | 5.02 | 17 |
| 17.95 | 4.94 | 35 |
| 18.47 | 4.80 | 5 |
| 18.84 | 4.71 | 25 |
| 19.08 | 4.65 | 5 |
| 20.65 | 4.30 | 6 |
| 21.24 | 4.18 | 10 |
| 21.52 | 4.13 | 21 |
| 22.14 | 4.01 | 37 |
| 22.76 | 3.90 | 11 |
| 22.85 | 3.89 | 12 |
| 23.41 | 3.80 | 4 |
| 23.91 | 3.72 | 8 |
| 24.16 | 3.68 | 12 |
| 24.50 | 3.63 | 29 |
| 24.73 | 3.60 | 13 |
| 24.91 | 3.57 | 4 |
| 25.67 | 3.47 | 25 |
| 26.36 | 3.38 | 100 |
| 26.62 | 3.35 | 14 |
| 27.10 | 3.29 | 4 |
| 27.63 | 3.23 | 4 |
| 28.20 | 3.16 | 17 |
| 28.62 | 3.12 | 2 |
| 29.18 | 3.06 | 10 |
| 30.17 | 2.96 | 2 |
| 30.47 | 2.93 | 8 |
| 30.76 | 2.90 | 11 |
| 32.00 | 2.79 | 9 |

### Example 12: Glycolic Acid Salt Crystalline Form 1

200.5 mg of 2-(5-methoxyisochroman-1-yl)-4,5-dihydro-1H-imidazole free base (I) was dissolved in 7 ml of ethanol, followed by vortexing until complete dissolution. 73.8 mg of glycolic acid was pre-dissolved in 1 ml of ethanol and was added over the free base solution to a 1:1 molar ratio of free base:acid. A clear solution was obtained. Subsequently, the anti-solvent (TBME) was added in 4 steps, at RT and under constant stirring. The total anti-solvent volume was 0.5, 1, 2 and 4 times the free base solution volume. Next, the sample was aged at 5° C. for 72 h. After completing the ageing time, the sample was decanted and the precipitate was air-dried at RT, in open vial, for 1 day. The resulting white powder was analyzed by XRPD. To confirm the salt form stability after complete drying, the sample was vacuum-dried (150 mbar, 30° C., 3 h) and re-analyzed by XRPD and was found to be a crystalline form 1 (table 12). The X-ray powder diffraction pattern of form 1 is depicted in FIG. 23 and the DSC thermogram in FIG. 24. DSC analysis shows an endotherm with an onset temperature approximately 167° C.

TABLE 12
X-ray powder reflections (up to 33° 2θ) and intensities
(normalized) of glycolic acid salt crystalline form 1. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 9.58 | 9.23 | 10 |
| 10.86 | 8.14 | 42 |
| 11.52 | 7.68 | 70 |
| 14.53 | 6.09 | 49 |
| 15.50 | 5.71 | 9 |
| 16.83 | 5.26 | 31 |
| 17.37 | 5.10 | 100 |
| 17.58 | 5.04 | 4 |
| 18.72 | 4.74 | 5 |
| 19.16 | 4.63 | 92 |
| 19.50 | 4.55 | 24 |
| 19.66 | 4.51 | 10 |
| 20.41 | 4.35 | 4 |
| 20.64 | 4.30 | 53 |
| 20.99 | 4.23 | 18 |
| 21.33 | 4.16 | 8 |
| 22.01 | 4.03 | 8 |
| 22.50 | 3.95 | 15 |
| 22.99 | 3.87 | 30 |
| 23.22 | 3.83 | 21 |
| 24.31 | 3.66 | 10 |
| 24.52 | 3.63 | 34 |
| 24.77 | 3.59 | 38 |
| 25.09 | 3.55 | 5 |
| 25.34 | 3.51 | 63 |
| 25.74 | 3.46 | 9 |
| 26.32 | 3.38 | 6 |
| 26.48 | 3.36 | 4 |
| 26.82 | 3.32 | 16 |
| 27.33 | 3.26 | 6 |
| 28.53 | 3.13 | 2 |
| 28.90 | 3.09 | 5 |
| 29.36 | 3.04 | 8 |
| 30.09 | 2.97 | 10 |
| 31.44 | 2.84 | 5 |

### Example 13: Glutaric Acid Salt Crystalline Form 1

200.3 mg of 2-(5-methoxyisochroman-1-yl)-4,5-dihydro-1H-imidazole free base (I) was dissolved in 10 ml of 2-propanol, followed by vortexing until complete dissolution. 126.4 mg of glutaric acid was dissolved in 1 ml of 2-propanol and was added over the free base solution to a 1:1 molar ratio of free base:acid. A clear solution was obtained. Subsequently, the anti-solvent (heptane) was added in 4 steps, at RT and under constant stirring. The total anti-solvent volume was 0.5, 1, 2 and 4 times the free base solution volume. Precipitation was observed. Next, the sample was aged at 5° C. for 72 h. After completing the ageing time, the sample was decanted and the precipitate was air-dried at RT, in open vial, for 1 day. The resulting white powder was analyzed by XRPD. To confirm the salt form stability after complete drying, the sample was vacuum-dried (150 mbar, 30° C., 3 h) and re-analyzed by XRPD and was found to be a crystalline form 1 (table 13). The X-ray powder diffraction pattern of form 1 is depicted in FIG. 25 and the DSC thermogram in FIG. 26. DSC analysis shows an endotherm with an onset temperature approximately 139° C.

TABLE 13
X-ray powder reflections (up to 33° 2θ) and intensities
(normalized) of glutaric acid salt crystalline form 1. The
value 2θ [°] represents the diffraction angle
in degrees and the value d [Å] represents the specified
distances in Å between the lattice planes.

| 2θ [°] | d [Å] | I/Iₒ [%] |
|---|---|---|
| 7.99 | 11.05 | 16 |
| 9.78 | 9.04 | 10 |
| 10.52 | 8.40 | 11 |
| 12.62 | 7.01 | 100 |
| 13.80 | 6.41 | 4 |
| 14.39 | 6.15 | 8 |
| 15.48 | 5.72 | 25 |
| 16.04 | 5.52 | 36 |
| 16.53 | 5.36 | 7 |
| 18.30 | 4.85 | 7 |
| 18.78 | 4.72 | 49 |
| 19.92 | 4.45 | 53 |
| 20.24 | 4.38 | 5 |
| 20.56 | 4.32 | 4 |
| 21.17 | 4.19 | 57 |
| 21.96 | 4.05 | 5 |
| 22.30 | 3.98 | 32 |
| 22.99 | 3.87 | 10 |
| 23.35 | 3.81 | 22 |
| 24.14 | 3.68 | 75 |
| 24.51 | 3.63 | 27 |
| 25.04 | 3.55 | 26 |
| 25.36 | 3.51 | 20 |
| 26.06 | 3.42 | 22 |
| 27.26 | 3.27 | 6 |
| 29.91 | 2.98 | 8 |

### Example 14: Adipic Acid Salt Crystalline Form 1

200.3 mg of 2-(5-methoxyisochroman-1-yl)-4,5-dihydro-1H-imidazole free base (I) was dissolved in 10 ml of 2-propanol, followed by vortexing until complete dissolution. 138.6 mg of adipic acid was pre-dissolved in 1 ml of 2-propanol by heating at 50° C. for 5 min and was added over the free base solution to a 1:1 molar ratio of free base:acid. A clear solution was obtained. Subsequently, the anti-solvent (heptane) was added in 4 steps, at RT and under constant stirring. The total anti-solvent volume was 0.5, 1, 2 and 4 times the free base solution volume. Precipitation was observed. Next, the sample was aged at 5° C. for 72 h. After completing the ageing time, the sample was decanted and the precipitate was air-dried at RT, in open vial, for 1 day. The resulting white powder was analyzed by XRPD. To confirm the salt form stability after complete drying, the sample was vacuum-dried (150 mbar, 30° C., 3 h) and re-analyzed by XRPD and was found to be a crystalline form 1 (table 14). The X-ray powder diffraction pattern of form 1 is depicted in FIG. 27 and the DSC thermogram in FIG. 28. DSC analysis shows an endotherm with an onset temperature approximately 131° C.

TABLE 14
X-ray powder reflections (up to 33° 2θ) and intensities
(normalized) of adipic acid salt crystalline form 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 7.69 | 11.48 | 6 |
| 9.73 | 9.08 | 4 |
| 10.50 | 8.42 | 38 |
| 12.09 | 7.32 | 63 |
| 12.43 | 7.11 | 9 |
| 13.75 | 6.43 | 3 |
| 14.44 | 6.13 | 13 |
| 15.13 | 5.85 | 40 |
| 15.40 | 5.75 | 33 |
| 17.80 | 4.98 | 38 |
| 18.25 | 4.86 | 52 |
| 19.57 | 4.53 | 13 |
| 19.96 | 4.44 | 100 |
| 20.32 | 4.37 | 5 |
| 20.60 | 4.31 | 17 |
| 21.02 | 4.22 | 63 |
| 21.95 | 4.05 | 11 |
| 22.32 | 3.98 | 35 |
| 22.82 | 3.89 | 25 |
| 23.14 | 3.84 | 24 |
| 23.40 | 3.80 | 26 |
| 24.24 | 3.67 | 48 |
| 24.67 | 3.61 | 32 |
| 25.00 | 3.56 | 50 |
| 25.61 | 3.48 | 11 |
| 26.55 | 3.35 | 4 |
| 27.13 | 3.28 | 11 |
| 27.57 | 3.23 | 4 |
| 27.90 | 3.20 | 5 |
| 28.61 | 3.12 | 4 |
| 30.02 | 2.97 | 10 |
| 31.09 | 2.87 | 10 |
| 31.63 | 2.83 | 6 |
| 32.37 | 2.76 | 3 |
| 32.67 | 2.74 | 6 |

### Example 15: Hydrobromic Acid Salt Crystalline Form 1

200.4 mg of 2-(5-methoxyisochroman-1-yl)-4,5-dihydro-1H-imidazole free base was dissolved in 12 ml of 2-propanol, followed by vortexing until complete dissolution. 110 μl of hydrobromic acid (47%) was added directly over the free base solution to a 1:1 molar ratio of free base:acid. A clear solution was obtained. Subsequently, the anti-solvent (heptane) was added in 4 steps, at RT and under constant stirring. The total anti-solvent volume was 0.5, 1, 2 and 4 times the free base solution volume. Next, the sample was aged at 5° C. for 24 h. After completing the ageing time, the sample was decanted and the precipitate was air-dried at RT for 6 days. The white powder was analyzed by XRPD. To confirm the salt form stability after complete drying, the sample was vacuum-dried (150 mbar, 30° C., 3 h) and re-analyzed by XRPD and was found to be a crystalline form 1 (table 15). The X-ray powder diffraction pattern of form 1 is depicted in FIG. 29 and the DSC thermogram in FIG. 30. DSC analysis shows two endotherms with onset temperatures approximately 59° C. and 130° C.

TABLE 15
X-ray powder reflections (up to 33° 2θ) and intensities (normalized)
of hydrobromic acid salt crystalline form 1. The value 2θ [°]
represents the diffraction angle in degrees and the value d [Å]
represents the specified distances in Å between the lattice planes.

| 2θ [°] | d [Å] | I/Iₒ [%] |
|---|---|---|
| 5.68 | 15.56 | 9 |
| 11.27 | 7.84 | 58 |
| 12.50 | 7.08 | 10 |
| 13.29 | 6.66 | 5 |
| 16.64 | 5.32 | 23 |
| 16.92 | 5.24 | 100 |
| 17.66 | 5.02 | 22 |
| 18.17 | 4.88 | 4 |
| 18.40 | 4.82 | 4 |
| 19.43 | 4.57 | 10 |
| 19.97 | 4.44 | 60 |
| 20.37 | 4.36 | 7 |
| 20.67 | 4.29 | 5 |
| 21.93 | 4.05 | 17 |
| 22.67 | 3.92 | 35 |
| 23.60 | 3.77 | 10 |
| 24.07 | 3.69 | 12 |
| 24.27 | 3.66 | 46 |
| 24.80 | 3.59 | 6 |
| 25.12 | 3.54 | 6 |
| 25.64 | 3.47 | 20 |
| 26.52 | 3.36 | 17 |
| 26.74 | 3.33 | 18 |
| 27.04 | 3.30 | 9 |
| 27.75 | 3.21 | 2 |
| 28.09 | 3.17 | 7 |
| 28.38 | 3.14 | 8 |
| 28.60 | 3.12 | 28 |
| 29.07 | 3.07 | 17 |
| 29.76 | 3.00 | 6 |
| 30.65 | 2.91 | 9 |
| 32.00 | 2.79 | 5 |
| 32.27 | 2.77 | 11 |

### Example 16: Mucic Acid Salt Crystalline Form 1

200.1 mg of 2-(5-methoxyisochroman-1-yl)-4,5-dihydro-1H-imidazole free base (I) was dissolved in 5 ml of ethanol, followed by vortexing until complete dissolution. 102.1 mg of mucic acid was pre-dissolved in 2.5 ml DMSO by heating at 50° C. for 5 min and was added over the free base solution to a 2:1 molar ratio of free base:acid. A clear solution was obtained. Subsequently, the anti-solvent (TBME) was added in 4 steps, at RT and under constant stirring. The total anti-solvent volume was 0.5, 1, 2 and 4 times the free base solution volume. Precipitation was observed. Next, the vial was aged at 5° C. for 72 h. After completing the ageing time, the sample was decanted and the precipitate was air-dried at RT for 1 day. The white powder was analyzed by XRPD. To confirm the salt form stability after complete drying, the sample was vacuum-dried (150 mbar, 30° C., 3 h) and re-analyzed by XRPD and was found to be a crystalline form 1 (table 16). The X-ray powder diffraction pattern of form 1 is depicted in FIG. 31 and the DSC thermogram in FIG. 32. DSC analysis shows several endotherms with onset temperatures approximately 33° C., 80° C. and 155° C.

TABLE 16
X-ray powder reflections (up to 33° 2θ) and intensities
(normalized) of mucic acid salt crystalline from 1. The value
2θ [°] represents the diffraction angle in degrees
and the value d [Å] represents the specified distances
in Å between the lattice planes.

| 2θ [°] | d [Å] | I/Iₒ [%] |
|---|---|---|
| 5.68 | 15.56 | 79 |
| 11.91 | 7.42 | 89 |
| 13.90 | 6.37 | 46 |
| 17.04 | 5.20 | 63 |
| 17.56 | 5.05 | 14 |
| 19.28 | 4.60 | 27 |
| 19.48 | 4.55 | 86 |
| 20.70 | 4.29 | 23 |
| 21.19 | 4.19 | 7 |
| 21.98 | 4.04 | 17 |
| 22.74 | 3.91 | 9 |
| 23.01 | 3.86 | 14 |
| 23.50 | 3.78 | 100 |
| 24.46 | 3.64 | 6 |
| 26.46 | 3.37 | 8 |
| 27.92 | 3.19 | 10 |
| 29.10 | 3.07 | 8 |

### Example 17:1-Hydroxy-2-Naphthoic Acid (Xianfoic Acid) Salt Crystalline Form 1

200.1 mg of 2-(5-methoxyisochroman-1-yl)-4,5-dihydro-1H-imidazole free base (I) was dissolved in 11 ml of 2-propanol, followed by vortexing until complete dissolution 182.1 mg of 1-hydroxy-2-naphthoic acid was dissolved in 6 ml of 2-propanol by heating at 50° C. for 5 min and was added over the free base solution to a 1:1 molar ratio of free base:acid. A clear solution was obtained. Subsequently, the anti-solvent (heptane) was added in 4 steps, at RT and under constant stirring. The total anti-solvent volume was 0.5, 1, 2 and 4 times the free base solution volume. A light precipitate was observed. Next, the vial was aged at 5° C. for 72 h following by solvent removal at RT, in open vial, for 5 h. For complete drying, the sample was vacuum-dried (200 mbar, 30° C., 48 h). The light-brown powder was analyzed by XRPD and was found to be a crystalline form 1 (table 17). The X-ray powder diffraction pattern of form 1 is depicted in FIG. 33 and the DSC thermogram in FIG. 34. DSC analysis shows an endotherm with an onset temperature approximately 120° C.

TABLE 17
X-ray powder reflections (up to 33° 2θ) and intensities (normalized)
of 1-hydroxy-2-naphtoic acid salt crystalline from 1. The value 2θ
[°] represents the diffraction angle in degrees and the value
d [Å] represents the specified distances in Å between the lattice planes.

| 2θ [°] | d [Å] | I/Iₒ [%] |
|---|---|---|
| 5.46 | 16.17 | 2 |
| 6.50 | 13.59 | 3 |
| 10.57 | 8.36 | 6 |
| 10.87 | 8.14 | 1 |
| 12.54 | 7.05 | 100 |
| 12.97 | 6.82 | 2 |
| 14.65 | 6.04 | 1 |
| 15.33 | 5.78 | 5 |
| 15.65 | 5.66 | 4 |
| 17.03 | 5.20 | 3 |
| 17.52 | 5.06 | 3 |
| 18.23 | 4.86 | 4 |
| 20.05 | 4.42 | 3 |
| 20.43 | 4.34 | 1 |
| 21.33 | 4.16 | 10 |
| 21.45 | 4.14 | 12 |
| 21.80 | 4.07 | 4 |
| 22.18 | 4.00 | 2 |
| 22.97 | 3.87 | 4 |
| 24.95 | 3.57 | 2 |
| 25.18 | 3.53 | 3 |
| 26.12 | 3.41 | 12 |
| 26.37 | 3.38 | 2 |
| 26.83 | 3.32 | 1 |
| 27.82 | 3.20 | 1 |

### Example 18: Hydrochloric Acid Salt Crystalline Form 1

200 mg of 2-(5-methoxyisochroman-1-yl)-4,5-dihydro-1H-imidazole free base (I) was dissolved in 3 ml of ethanol. Hydrochloric acid 35% aq. (82.3 μl) was added directly over the free base solution to a 1:1 molar ratio of free base:acid. A clear solution was obtained. The resulting solution was heated at 60° C. for 30 min. Next, the solution was cooled at RT during 2 h and aged at 5° C. for 24 h. After completing the ageing time, a clear solution was obtained and solvent removal was achieved at RT, in open vial, for 5 days. The light-yellow powder (crystals) was analyzed by XRPD. To confirm the salt form stability after complete drying, the sample was vacuum-dried (150 mbar, 30° C., 3 h) and re-analyzed by XRPD and was found to be a crystalline form 1 (table 18). The X-ray powder diffraction pattern of form 1 is depicted in FIG. 35 and the DSC thermogram in FIG. 36. DSC analysis shows an endotherm with an onset temperature approximately 87° C.

TABLE 18
X-ray powder reflections (up to 33° 2θ) and intensities
(normalized) of hydrocholoric acid salt crystalline from 1.
The value 2θ [°] represents the diffraction angle
in degrees and the value d [Å] represents the specified
distances in Å between the lattice planes.

| 2θ [°] | d [Å] | I/Iₒ [%] |
|---|---|---|
| 5.50 | 16.04 | 8 |
| 10.73 | 8.24 | 48 |
| 10.98 | 8.05 | 38 |
| 12.60 | 7.02 | 79 |
| 13.53 | 6.54 | 22 |
| 14.05 | 6.30 | 6 |
| 16.43 | 5.39 | 100 |
| 17.66 | 5.02 | 11 |
| 18.76 | 4.73 | 2 |
| 19.78 | 4.48 | 4 |
| 20.50 | 4.33 | 15 |
| 20.66 | 4.30 | 12 |
| 21.04 | 4.22 | 5 |
| 21.95 | 4.05 | 23 |
| 22.41 | 3.96 | 62 |
| 23.58 | 3.77 | 5 |
| 24.20 | 3.67 | 24 |
| 25.23 | 3.53 | 17 |
| 25.64 | 3.47 | 2 |
| 25.96 | 3.43 | 10 |
| 27.23 | 3.27 | 6 |
| 27.92 | 3.19 | 2 |
| 28.24 | 3.16 | 4 |
| 28.68 | 3.11 | 4 |
| 28.90 | 3.09 | 33 |
| 29.81 | 2.99 | 2 |
| 31.01 | 2.88 | 4 |
| 31.28 | 2.86 | 16 |

### Experiment 1. Physical Stability of the Salts

A stability test was carried out for all crystalline salt forms resulted from the salt screen by exposing the solids for 2 or 3 weeks at 40° C. and 70-80% relative humidity. After exposure, the solids were re-analyzed by XRPD, followed by physical stability assessment. The results are shown in table 19.

TABLE 19
Physical stability of the salts of compound (I)

| | | Physical | |
|---|---|---|---|
| Salt form of compound (I) | Form | stability | Form after stability |
| Naphthalene-1,5-disulfonic | Form 1 | Stable | Form 1 |
| acid salt | | | |
| Ethane-1,2-disulfonic | Form 1 | Stable | Form 1 |
| acid salt | | | |
| Oxalic acid salt | Form 1 | Stable | Form 1 |
| Saccharin salt | Form 1 | Partially | Form 1 + Form 2 |
| | | stable | |
| Saccharin salt | Form 2 | Stable | Form 2 |
| Maleic acid salt | Form 1 | Stable | Form 1 |
| Malonic acid salt | Form 1 | Stable | Form 1 |
| L-tartaric acid salt | Form 1 | Stable | Form 1 |
| L-tartaric acid salt | Form 2 | Unstable | Form 1 |
| Fumaric acid salt | Form 1 | Stable | Form 1 |
| Fumaric acid salt | Form 2 | Stable | Form 2 |
| Glycolic acid salt | Form 1 | Stable | Form 1 |
| Glutaric acid salt | Form 1 | Stable | Form 1 |
| Adipic acid salt | Form 1 | Stable | Form 1 |
| Hydrobromic acid salt | Form 1 | Stable | Form 1 |
| Mucic acid salt | Form 1 | Stable | Form 1 |
| 1-Hydroxy-2-napthoic | Form 1 | Stable | Form 1 |
| acid salt | | | |
| Hydrochloric acid salt | Form 1 | Stable | Form 1 |
| Methanesulfonic acid salt | Form 1 | Unstable | Amorphous |
| Cyclamic acid salt | Form 1 | Unstable | Form 2 |
| Cyclamic acid salt | Form 2 | Partially | Form 2 + extra |
| | | stable | peaks |
| L-Glutamic acid salt | Form 1 | Partially | Form 1 + extra |
| | | stable | peaks |
| D,L-Lactic acid salt | Form 1 | Unstable | Form 2 |
| 2-Ketoglutaric acid salt | Form 1 | Unstable | Amorphous |
| L-Aspartic acid salt | Form 1 | Unstable | Form 1 + amorphous |

The results show that sixteen salt forms remained stable after storage at elevated temperature and relative humidity; salts with naphthalene-1,5-disulfonic acid, ethane-1,2-disulfonic acid, oxalic acid, saccharin (form 2), maleic acid, malonic acid, L-tartaric acid (form 1), fumaric acid (forms 1 and 2), glycolic acid, glutaric acid, adipic acid, hydrobromic acid, mucic acid, 1-hydroxy-2-napthoic acid and hydrochloric acid. Salts with cyclamic acid (form 2) and L-glutamic acid showed extra peaks in the XRPD pattern, one form changed partially (a salt with saccharin (form 1)) or completely (salts with L-tartaric acid (form 2), cyclamic acid (form 1) and D,L-lactic acid) into more stable forms. Two salts turned into amorphous phase (salts with methanesulfonic acid and 2-ketoglutaric acid) while the salt with L-aspartic acid showed an amorphous phase with characteristic peaks of the counter-ion.

### Experiment 2: Chemical Stability of the Salts

The chemical stability of various salts of compound (I) was determined by stressing the salts for 1 and 14 days at 60° C. and 75% relative humidity. The unstressed sample was used as an reference. The purity of the samples were analyzed using UHPLC, and the purity difference between the stressed and reference was calculated. The results are shown in table 20.

TABLE 20
Physical stability of the salts of compound (I)

| | | | | | Difference |
|---|---|---|---|---|---|
| | | | Difference | | between |
| | | | between | Purity (%) | reference and |
| | | Purity (%) of | reference and | of 14 day | 14 day |
| Sample (salt form of | Purity (%) | 1 day stress | 1 day stressed | stress | stressed |
| compound (I)) | of reference | samples | sample* | samples | sample* |
| Hydrochloric acid salt | 98.6 | 89.8 | 8.7 | 69.0 | 29.5 |
| Oxalic acid salt | 97.7 | 97.9 | −0.2 | 97.9 | −0.2 |
| Napthalene-1,5- | 96.3 | 96.1 | 0.2 | 95.7 | 0.6 |
| disulfonic acid salt | | | | | |
| Ethane-1,2-disulfonic | 99.8 | 99.8 | 0.0 | 99.8 | 0.0 |
| acid salt | | | | | |
| Saccharin salt | 97.1 | 95.0 | 2.1 | 91.5 | 5.6 |
| Maleic acid salt | 99.6 | 64.0 | 35.7 | 33.3 | 66.4 |
| Malonic acid salt | 98.2 | 96.6 | 1.5 | 72.7 | 25.5 |
| L-tartaric acid salt | 99.0 | 97.9 | 1.0 | 90.3 | 8.7 |
| (form 1) | | | | | |
| L-tartaric acid salt | 98.6 | 97.5 | 1.0 | 88.3 | 10.3 |
| (form 2) | | | | | |
| Fumaric acid salt | 97.2 | 97.6 | −0.3 | 98.0 | −0.8 |
| (form 2) | | | | | |
| Glycolic acid salt | 99.8 | 99.3 | 0.5 | 17.6 | 82.2 |
| Glutaric acid salt | 96.4 | 78.7 | 17.7 | 2.0 | 94.4 |
| Adipic acid salt | 99.1 | 94.7 | 4.4 | 2.7 | 96.5 |
| Hydrobromic acid salt | 98.3 | 98.1 | 0.2 | 98.2 | 0.1 |
| Mucic acid salt | 99.2 | 97.7 | 1.6 | 35.6 | 63.6 |
| 1-Hydroxy-2-naphtoic | 98.9 | 95.4 | 3.5 | 0.6 | 98.3 |
| acid salt | | | | | |

*Calculated using more accurate values than shown in the table.

The results show that five salt forms have better stability with a degradation of less than 1.5% in 14 days compared to the reference. These are salts with oxalic acid, napthalene-1,5-disulfonic acid, ethane-1,2-disulfonic acid, fumaric acid and hydrobromic acid.

A person skilled in the art will appreciate that the embodiments described herein can be modified without departing from the inventive concept. A person skilled in the art also understands that the present disclosure is not limited to the particular embodiments disclosed but is intended to also cover modifications of the embodiments that are within the scope of the present disclosure.

1. A salt of 2-(5-methoxyisochroman-1-yl)-4,5-dihydro-1H-imidazole (I) with an acid selected from the group consisting of naphthalene-1,5-disulfonic acid, ethane-1,2-disulfonic acid, oxalic acid, saccharin, maleic acid, malonic acid, L-tartaric acid, fumaric acid, glycolic acid, glutaric acid, adipic acid, hydrobromic acid, mucic acid, 1-hydroxy-2-napthoic acid and hydrochloric acid.

2. The salt according to claim 1, which is a salt of 2-(5-methoxyisochroman-1-yl)-4,5-dihydro-1H-imidazole (I) with an acid selected from the group consisting of naphthalene-1,5-disulfonic acid, ethane-1,2-disulfonic acid, oxalic acid, saccharin, maleic acid, malonic acid, L-tartaric acid, glycolic acid, glutaric acid, adipic acid, hydrobromic acid, mucic acid and 1-hydroxy-2-napthoic acid.

3. The salt according to claim 1, which is a salt of 2-(5-methoxyisochroman-1-yl)-4,5-dihydro-1H-imidazole (I) with an acid selected from the group consisting of napthalene-1,5-disulfonic acid, ethane-1,2-disulfonic acid, oxalic acid and hydrobromic acid.

4. The salt according to claim 1, which is crystalline.

5. The salt according to claim 1, which is a crystalline napthalene-1,5-disulfonic acid salt of 2-(5-methoxyisochroman-1-yl)-4,5-dihydro-1H-imidazole (I).

6. The salt according to claim 5, which is crystalline form characterized by an X-ray powder diffraction pattern comprising peaks at 18.6±0.2, 19.1±0.2, 21.5±0.2, 22.4±0.2 and 23.9±0.2 degrees 2-theta.

7. The salt according to claim 1, which is a crystalline ethane-1,2-disulfonic acid salt of 2-(5-methoxyisochroman-1-yl)-4,5-dihydro-1H-imidazole (I).

8. The salt according to claim 7, which is crystalline form characterized by an X-ray powder diffraction pattern comprising peaks at 13.5±0.2, 14.5±0.2, 16.0±0.2, 20.3±0.2 and 22.8±0.2 degrees 2-theta.

9. The salt according to claim 1, which is a crystalline oxalic acid salt of 2-(5-methoxyisochroman-1-yl)-4,5-dihydro-1H-imidazole (I).

10. The salt according to claim 9, which is crystalline form characterized by an X-ray powder diffraction pattern comprising peaks at 14.3±0.2, 17.0±0.2, 17.6±0.2, 19.8±0.2, 22.3±0.2 and 23.5±0.2 degrees 2-theta.

11. The salt according to claim 1, which is a crystalline saccharin salt of 2-(5-methoxyisochroman-1-yl)-4,5-dihydro-1H-imidazole (I).

12. The salt according to claim 11, which is crystalline form characterized by an X-ray powder diffraction pattern comprising peaks at 14.1±0.2, 15.1±0.2, 15.8±0.2, 17.7±0.2 and 19.1±0.2 degrees 2-theta.

13. The salt according to claim 1, which is a crystalline maleic acid salt of 2-(5-methoxyisochroman-1-yl)-4,5-dihydro-1H-imidazole (I).

14. The salt according to claim 13, which is crystalline form characterized by an X-ray powder diffraction pattern comprising peaks at 10.2±0.2, 11.6±0.2, 16.8±0.2, 17.4±0.2 and 19.3±0.2 degrees 2-theta.

15. The salt according to claim 1, which is a crystalline malonic acid salt of 2-(5-methoxyisochroman-1-yl)-4,5-dihydro-1H-imidazole (I).

16. The salt according to claim 15, which is crystalline form characterized by an X-ray powder diffraction pattern comprising peaks at 10.1±0.2, 10.8±0.2, 14.1±0.2, 16.3±0.2 and 17.4±0.2 degrees 2-theta.

17. The salt according to claim 1, which is a crystalline L-tartaric acid salt of 2-(5-methoxyisochroman-1-yl)-4,5-dihydro-1H-imidazole (I).

18. The salt according to claim 17, which is crystalline form characterized by an X-ray powder diffraction pattern comprising peaks at 11.8±0.2, 12.5±0.2, 12.9±0.2, 13.4±0.2 and 14.0±0.2 degrees 2-theta.

19. The salt according to claim 1, which is a crystalline fumaric acid salt of 2-(5-methoxyisochroman-1-yl)-4,5-dihydro-1H-imidazole (I).

20. The salt according to claim 19, which is crystalline form characterized by an X-ray powder diffraction pattern comprising peaks at 11.3±0.2, 13.2±0.2, 15.5±0.2, 22.5±0.2, 24.2±0.2 and 25.3±0.2 degrees 2-theta.

21. The salt according to claim 19, which is crystalline form characterized by an X-ray powder diffraction pattern comprising peaks at 10.6±0.2, 15.9±0.2, 18.0±0.2, 22.1±0.2 and 26.4±0.2 degrees 2-theta.

22. The salt according to claim 1, which is a crystalline glycolic acid salt of 2-(5-methoxyisochroman-1-yl)-4,5-dihydro-1H-imidazole (I).

23. The salt according to claim 22, which is crystalline form characterized by an X-ray powder diffraction pattern comprising peaks at 11.5±0.2, 14.5±0.2, 17.4±0.2, 19.2±0.2, 20.6±0.2 and 25.3±0.2 degrees 2-theta.

24. The salt according to claim 1, which is a crystalline glutaric acid salt of 2-(5-methoxyisochroman-1-yl)-4,5-dihydro-1H-imidazole (I).

25. The salt according to claim 24, which is crystalline form characterized by an X-ray powder diffraction pattern comprising peaks at 12.6±0.2, 16.0±0.2, 18.8±0.2, 19.9±0.2, and 21.2±0.2 degrees 2-theta.

26. The salt according to claim 1, which is a crystalline adipic acid salt of 2-(5-methoxyisochroman-1-yl)-4,5-dihydro-1H-imidazole (I).

27. The salt according to claim 26, which is crystalline form characterized by an X-ray powder diffraction pattern comprising peaks at 10.5±0.2, 12.1±0.2, 15.1±0.2, 18.3±0.2, 20.0±0.2 and 21.0±0.2 degrees 2-theta.

28. The salt according to claim 1, which is a crystalline hydrobromic acid salt of 2-(5-methoxyisochroman-1-yl)-4,5-dihydro-1H-imidazole (I).

29. The salt according to claim 28, which is crystalline form characterized by an X-ray powder diffraction pattern comprising peaks at 11.3±0.2, 16.9±0.2, 20.0±0.2, 22.7±0.2 and 24.3±0.2 degrees 2-theta.

30. The salt according to claim 1, which is a crystalline mucic acid salt of 2-(5-methoxyisochroman-1-yl)-4,5-dihydro-1H-imidazole (I).

31. The salt according to claim 30, which is crystalline form characterized by an X-ray powder diffraction pattern comprising peaks at 5.7±0.2, 11.9±0.2, 13.9±0.2, 17.0±0.2, 19.5±0.2 and 23.5±0.2 degrees 2-theta.

32. The salt according to claim 1, which is a crystalline 1-hydroxy-2-napthoic acid salt of 2-(5-methoxyisochroman-1-yl)-4,5-dihydro-1H-imidazole (I).

33. The salt according to claim 32, which is crystalline form characterized by an X-ray powder diffraction pattern comprising peaks at 10.6±0.2, 12.5±0.2, 15.3±0.2, 18.2±0.2 and 26.1±0.2 degrees 2-theta.

34. The salt according to claim 1, which is a crystalline hydrochloric acid salt of 2-(5-methoxyisochroman-1-yl)-4,5-dihydro-1H-imidazole (I).

35. The salt according to claim 34, which is crystalline form characterized by an X-ray powder diffraction pattern comprising peaks at 10.7±0.2, 11.0±0.2, 12.6±0.2, 16.4±0.2 and 22.4±0.2 degrees 2-theta.

36. A method of preparing the crystalline salt according to claim 5, comprising dissolving compound (I) and napthalene-1,5-disulfonic acid in acetone, adding water, cooling the mixture, and isolating the crystalline product.

37. A method of preparing the crystalline salt according to claim 7, comprising dissolving compound (I) and ethane-1,2-disulfonic acid in 2-propanol, adding heptane, cooling the mixture, and isolating the crystalline product.

38. A method of preparing the crystalline salt according to claim 9, comprising dissolving compound (I) and oxalic acid in acetone, cooling the mixture, and isolating the crystalline product.

39. A method of preparing the crystalline salt according to claim 28, comprising dissolving compound (I) in 2-propanol follow by addition of hydrobromic acid, adding heptane, cooling the mixture, and isolating the crystalline product.

40. A pharmaceutical composition comprising a salt of 2-(5-methoxyisochroman-1-yl)-4,5-dihydro-1H-imidazole (I) with an acid selected from the group consisting of naphthalene-1,5-disulfonic acid, ethane-1,2-disulfonic acid, oxalic acid, saccharin, maleic acid, malonic acid, L-tartaric acid, fumaric acid, glycolic acid, glutaric acid, adipic acid, hydrobromic acid, mucic acid, 1-hydroxy-2-napthoic acid and hydrochloric acid as an active ingredient together with one or more excipients.

41. A method of treating anxiety comprising administering a salt of 2-(5-methoxyisochroman-1-yl)-4,5-dihydro-1H-imidazole (I) with an acid selected from the group consisting of naphthalene-1,5-disulfonic acid, ethane-1,2-disulfonic acid, oxalic acid, saccharin, maleic acid, malonic acid, L-tartaric acid, fumaric acid, glycolic acid, glutaric acid, adipic acid, hydrobromic acid, mucic acid, 1-hydroxy-2-napthoic acid and hydrochloric acid to a subject in need thereof.

42. The pharmaceutical composition according to claim 40, wherein the salt is a crystalline napthalene-1,5-disulfonic acid salt of 2-(5-methoxyisochroman-1-yl)-4,5-dihydro-1H-imidazole (I).

43. The method of treating anxiety according to claim 41, wherein the salt is a crystalline napthalene-1,5-disulfonic acid salt of 2-(5-methoxyisochroman-1-yl)-4,5-dihydro-1H-imidazole (I).

44. The pharmaceutical composition according to claim 40, wherein the salt is a crystalline ethane-1,2-disulfonic acid salt of 2-(5-methoxyisochroman-1-yl)-4,5-dihydro-1H-imidazole (I).

45. The method of treating anxiety according to claim 41, wherein the salt is a crystalline ethane-1,2-disulfonic acid salt of 2-(5-methoxyisochroman-1-yl)-4,5-dihydro-1H-imidazole (I).

46. The pharmaceutical composition according to claim 40, wherein the salt is a crystalline oxalic acid salt of 2-(5-methoxyisochroman-1-yl)-4,5-dihydro-1H-imidazole (I).

47. The method of treating anxiety according to claim 41, wherein the salt is a crystalline oxalic acid salt of 2-(5-methoxyisochroman-1-yl)-4,5-dihydro-1H-imidazole (I).

48. The pharmaceutical composition according to claim 40, wherein the salt is a crystalline hydrobromic acid salt of 2-(5-methoxyisochroman-1-yl)-4,5-dihydro-1H-imidazole (I).

49. The method of treating anxiety according to claim 41, wherein the salt is a crystalline hydrobromic acid salt of 2-(5-methoxyisochroman-1-yl)-4,5-dihydro-1H-imidazole (I).
